SEQUENCE LISTING (7 sequences)

SEQ ID NO: 1 - DNA, 40 bp, Artificial
  primer
ggagagtctggcctggagaatcccacacttgtcaatagcc

SEQ ID NO: 2 - DNA, 28 bp, Artificial
  primer
atcaagcgtttcctggaggacaccacgg

SEQ ID NO: 3 - DNA, 25 bp, Artificial
  primer
ctagtggcagccctgcccctggtgc

SEQ ID NO: 4 - DNA, 38 bp, Artificial
  primer
tcgaggatccatcaagcgtttcctggaggacaccacgg

SEQ ID NO: 5 - DNA, 24 bp, Artificial
  primer
ctatgccacaggcttccagcactc

SEQ ID NO: 6 - protein, 274 aa, Homo sapiens
MIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGKSTLVNSLFLTDLYRDRKLLGAEERIMQTVEITKHAVDIEEKGVRLRLTIVDTPGFGDAVNNTECWKPVAEYIDQQFEQYFRDESGLNRKNIQDNRVHCCLYFISPFGHGYGPSLRLLAPPGAVKGTGQEHQGQGCH

SEQ ID NO: 7 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

(19) United States
(12) Patent Application Publication
(10) Pub. No.: US 2005/0227302 A1
(43) Pub. Date: Oct. 13, 2005

(54) Induction of apoptosis via ARTS-IAP complexes

(76) Inventors: Sarit Larisch, Neve Habaron (IL); Yossi Gottfried, Kiryat Tivon (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 11/028,509

(22) Filed: Jan. 4, 2005

Related U.S. Application Data
(60) Provisional application No. 60/534,389, filed on Jan. 7, 2004.

(51) Int. Cl. A61K 38/54; C12Q 1/68; G01N 33/574

(52) U.S. Cl. 435/7.23; 514/2

(57) ABSTRACT

The present invention provides complexes containing an ARTS protein and an IAP protein, compounds that disrupt same, and use of the compounds in treating neuro-degenerative disease, ischemic injury, myelodysplasia, atherosclerosis, various auto-immune diseases, cytopenia, pancreatitis, and periodonitis, and in decreasing susceptibility of a cell to apoptosis. The present invention also provides methods for identifying biologically active regions of ARTS protein, and use of mimetic compounds of same in treating apoptosis-related disorders, cancer, and other neoplastic diseases and disorders.

### CROSS REFERENCE TO RELATED APPLICATION

This application claims priority of U.S. Provisional Application Ser. No. 60/534,389, filed Jan. 7, 2004, which is hereby incorporated herein in its entirety by reference.

### FIELD OF INVENTION

The present invention provides complexes containing an ARTS protein and an IAP protein, compounds that disrupt same, and use of the compounds in treating neuro-degenerative disease, ischemic injury, myelodysplasia, atherosclerosis, various auto-immune diseases, cytopenia, pancreatitis, and periodonitis, and in decreasing susceptibility of a cell to apoptosis. The present invention also provides methods for identifying biologically active regions of ARTS protein, and use of mimetic compounds of same in treating apoptosis-related disorders, cancer, and other neoplastic diseases and disorders.

### BACKGROUND OF THE INVENTION

Programmed cell death by apoptosis is a major mechanism for regulating cell number and tissue homeostasis. Apoptosis is tightly controlled through the action of both activators and inhibitors of caspases. One family of caspase inhibitors is the Inhibitors of Apoptosis Proteins (IAPs), All IAP proteins contain between one to three baculoviral IAP repeat (BIR) domains, which directly interact with caspases and inhibit their apoptotic activity XIAP, one member of the IAP protein family, can directly inhibit caspases 3, 7 and 9.

ARTS is a pro-apoptotic protein derived by differential splicing from the human septin H5/PNUTL2/CDCrel-2a (Sept4) gene. ARTS contains a P-loop GTP-binding motif conserved in the Sept family. Yet unlike most other Sept family members, it is localized to mitochondria and promotes apoptosis via TGF-beta and other pro-apoptotic stimuli, such as etoposide, arabinoside (ara-C), staurosporine and Fas. A number of types of cancer and neoplastic cells lack ARTS protein expression and/or activity.

Methods for inducing apoptosis in cells, for example neoplastic or cancer cells, are needed for therapeutic applications for a wide range of diseases.

### SUMMARY OF THE INVENTION

In one embodiment, the present invention provides a method of identifying a region of an ARTS protein or a variant or homologue thereof that mediates a biological activity, comprising the steps of: (a) generating a subset of test fragments of the ARTS protein that bind to an IAP protein or a variant or homologue thereof; and (b) analyzing the subset for inclusion of a common region, whereby the common region defines a region of an ARTS protein or a variant or homologue thereof that mediates a biological activity

In another embodiment, the present invention provides a mimetic compound of the common region identified by one of the methods of the identifying a region of an ARTS protein or a variant or homologue thereof that mediates a biological activity.

In another embodiment, the present invention provides a method of inducing an apoptosis, a killing of a cancer cell, or in treating: a psoriasis, a tuberculosis infection, a Bartonella infection, a vitiligo, an atopic dermatitis, a hyper-proliferative or UV-responsive dermatosis, or a lymphohistiocytosis, comprising administering the mimetic compound of the present invention.

In another embodiment, the present invention provides a method of identifying a compound useful for a chemotherapy of a neoplastic disease or disorder, comprising testing the mimetic compound of the present invention for an activity against the neoplastic disease or disorder, whereby, if the mimetic compound exhibits an activity against the neoplastic disease or disorder, then the mimetic compound is a compound useful for a chemotherapy of a neoplastic disease or disorder.

In another embodiment, the present invention provides a method of identifying a region of an ARTS protein or a variant or homologue thereof that mediates a biological activity, comprising the steps of: (a) generating a subset of test fragments of the ARTS protein, variant, or homologue thereof that reduce a level of an LAP protein or a variant or homologue thereof; and (b) analyzing the subset for inclusion of a common region, whereby the common region defines a region of an ARTS protein or a variant or homologue thereof that mediates a biological activity.

In another embodiment, the present invention provides a method of identifying a region of an ARTS protein or a variant or homologue thereof that mediates a biological activity, comprising the steps of: (a) generating a subset of test fragments of the ARTS protein that induce the translocation of an IAP protein, variant, or homologue thereof; and (b) analyzing the subset for inclusion of a common region, whereby the common region defines a region of an ARTS protein or a variant or homologue thereof that mediates a biological activity.

In another embodiment, the present invention provides a method of inducing apoptosis in a cell, comprising the step of inhibiting or reducing a degradation of an ARTS protein or a variant or homologue thereof.

In another embodiment, the present invention provides an isolated complex, comprising an ARTS protein or a variant or homologue thereof and an IAP protein or a variant or homologue thereof.

In another embodiment, the present invention provides a compound that selectively binds to the isolated complex of the present invention.

In another embodiment, the present invention provides a compound that inhibits a formation of the isolated complex of the present invention.

In another embodiment, the present invention provides a compound that disrupts the isolated complex of the present invention.

In another embodiment, the present invention provides a method of testing a compound for an ability to inhibit or reduce an incidence of an apoptosis, comprising determining whether the compound disrupts the isolated complex of the present invention, whereby if the compound disrupts the isolated complex of the present invention, then the compound inhibits or reduces an incidence of an apoptosis.

In another embodiment, the present invention provides a method of treating or reducing an incidence of a neuro-degenerative disease, comprising administering a compound that disrupts the isolated complex of the present invention, thereby treating or reducing an incidence of a neuro-degenerative disease.

In another embodiment, the present invention provides a method of treating or reducing an incidence of an ischemic injury, comprising administering a compound that disrupts the isolated complex of the present invention, thereby treating or reducing an incidence of an ischemic injury.

In another embodiment, the present invention provides a method of treating or reducing an incidence of a myelodysplasia, comprising administering a compound that disrupts the isolated complex of the present invention, thereby treating or reducing an incidence of a myelodysplasia.

In another embodiment, the present invention provides a method of treating or reducing an incidence of an atherosclerosis, comprising administering a compound that disrupts the isolated complex of the present invention, thereby treating or reducing an incidence of an atherosclerosis.

In another embodiment, the present invention provides a method of treating or reducing an incidence of an auto-immune disease, comprising administering a compound that disrupts the isolated complex of the present invention, thereby treating or reducing an incidence of an auto-immune disease.

In another embodiment, the present invention provides a method of treating or reducing an incidence of a Crohn's disease or ulcerative colitis, comprising administering a compound that disrupts the isolated complex of the present invention, thereby treating or reducing an incidence of a Crohn's disease or ulcerative colitis.

In another embodiment, the present invention provides a method of treating or reducing an incidence of a cytopenia, comprising administering a compound that disrupts the isolated complex of the present invention, thereby treating or reducing an incidence of a cytopenia.

In another embodiment, the present invention provides a method of treating or reducing an incidence of a pancreatitis, comprising administering a compound that disrupts the isolated complex of the present invention, thereby treating or reducing an incidence of a pancreatitis.

In another embodiment, the present invention provides a method of treating or reducing an incidence of a periodontitis, comprising administering a compound that disrupts the isolated complex of the present invention, thereby treating or reducing an incidence of a periodontitis.

In another embodiment, the present invention provides a method of testing a compound for an ability to inhibit or reduce an incidence of an apoptosis, comprising determining whether the compound prevents a binding of an ARTS protein or a variant or homologue thereof to an XIAP protein or a variant or homologue thereof, whereby if the compound prevents binding of the ARTS protein, variant or homologue thereof, to the IAP protein, variant, or homologue thereof, then the compound inhibits or reduces an incidence of an apoptosis.

In another embodiment, the present invention provides a method of treating or reducing an incidence of a neuro-degenerative disease, comprising administering a compound that prevents a binding of an ARTS protein or a variant or homologue thereof to an XIAP protein or a variant or homologue thereof, thereby treating or reducing an incidence of a neuro-degenerative disease.

In another embodiment, the present invention provides a method of treating of reducing an incidence of an ischemic injury, comprising administering a compound that prevents a binding of an ARTS protein or a variant or homologue thereof to an XIAP protein or a variant or homologue thereof, thereby treating or reducing an incidence of an ischemic injury.

In another embodiment, the present invention provides a method of treating or reducing an incidence of a myelodysplasia, comprising administering a compound that prevents a binding of an ARTS protein or a variant or homologue thereof to an XIAP protein or a variant or homologue thereof, thereby treating or reducing an incidence of a myelodysplasia.

In another embodiment, the present invention provides a method of treating or reducing an incidence of an atherosclerosis, comprising administering a compound that prevents a binding of an ARTS protein or a variant or homologue thereof to an XIAP protein or a variant or homologue thereof, thereby treating or reducing an incidence of an atherosclerosis.

In another embodiment, the present invention provides a method of treating or reducing an incidence of an auto-immune disease, comprising administering a compound that prevents a binding of an ARTS protein or a variant or homologue thereof to an XIAP protein or a variant or homologue thereof, thereby treating or reducing an incidence of an auto-immune disease.

In another embodiment, the present invention provides a method of treating or reducing an incidence of a Crohn's disease or ulcerative colitis, comprising administering a compound that prevents a binding of an ARTS protein or a variant or homologue thereof to an XIAP protein or a variant or homologue thereof, thereby treating or reducing an incidence of a Crohn's disease or ulcerative colitis.

In another embodiment, the present invention provides a method of treating or reducing an incidence of a cytopenia, comprising administering a compound that prevents a binding of an ARTS protein or a variant or homologue thereof to an XIAP protein or a variant or homologue thereof, thereby treating or reducing an incidence of a cytopenia.

In another embodiment, the present invention provides a method of treating or reducing an incidence of a pancreatitis, comprising administering a compound that prevents a binding of an ARTS protein or a variant or homologue thereof to an XIAP protein or a variant or homologue thereof, thereby treating or reducing an incidence of a pancreatitis.

In another embodiment, the present invention provides a method of treating or reducing an incidence of a periodontitis, comprising administering a compound that prevents a binding of an ARTS protein or a variant or homologue thereof to an XIAP protein or a variant or homologue thereof, thereby treating or reducing an incidence of a periodontitis.

### BRIEF DESCRIPTION OF THE FIGURES

FIG. 1: ARTS induces caspase 3 activity in response to variety of apoptotic inducers, and caspase activity affects ARTS localization. Cells were transiently transfected with either control vector or the AU5-ARTS construct and treated with various apoptotic inducers. A. ARTS induces caspase 3 activity in response to different apoptotic inducers. A549 cells were treated with TGF-β, K562 and HL-60 with ara-C. Caspase activity is presented as the fold-increase compared to cells transfected with control vector without apoptotic treatment. P-values (Anova) of K562 and HL-60 cells were 0.02 and 0.001, respectively; A549 p-value (Anova) was 0.09. B. COS-7 cells were treated with 50 mM etoposide for 16 hours with and without BOC. P-value (Anova) was 0.0026. C. Immunofluorescence assay of COS-7 cells with anti-mitochondria and anti-ARTS antibodies. Cells were treated with 50 μM etoposide for 16 hour without and with BOC (caspase inhibitor). Cells treated with etoposide alone showed typical morphological changes associated with apoptosis and accumulated ARTS in the nucleus (panels I and II) (mitochondrial border shown by a thin white line; merged mitochondria-ARTS fluorescence shown by cross-hatching). Upon addition of caspase inhibitors nuclear translocation of ARTS was blocked, although peri-nuclear clustering of ARTS-positive mitochondria was still observed (panels III and IV).

FIG. 2: Mutations in Drosophila peanut dominantly suppress cell killing induced by Reaper and Hid Reducing the amount of peanut by 50% (in pnut1/+ or pnutXP/+ heterozygous animals) significantly increased the eye size of both GMR-Hid and GMR-Reaper flies compared to a control background (+/+). Under the same conditions, reducing the dosage of the Drosophila Apaf-1 homolog (hac-1/+) had very little effect.

FIG. 3. ARTS specifically binds XIAP in vitro. In vitro binding was tested using a GST pull-down assay. A-B. COS-7 cells were transiently transfected with myc-tagged XIAP. GST pull-down was performed using GST-ARTS and Western blot using anti-XIAP and (A) and anti myc (B). C. COS-7 cells were transiently transfected with AU5-ARTS, AU5-ARTSDC or AU5-H5. GST pull down was performed using GST-XIAP and Western blot with anti-AU5. Binding to glutathione beads alone served as a negative control.

FIG. 4. ARTS binding to XIAP is specific and related to its apoptotic function. A. In-vivo binding of ARTS to XIAP was tested using co-IP with anti-ARTS, anti-XIAP or mouse IgG as a negative control, followed by anti-XIAP Western blot. B. Lysates of COS-7 cells co-transfected with pcDNA-mycXIAP and AU5-ARTS, AU5-ARTSmGTP or H5 DC were co-immunoprecipitated using agarose-anti-myc beads, followed by Western blot analysis with rabbit anti-myc and anti-ARTS. Mutant forms of ARTS, as well as H5, did not bind to XIAP C. COS-7 cells were transiently transfected with AU5-ARTS, AU5-ARTSmGTP, AU5-ARTSDC or AU5-PNUTL2, and percent apoptosis determined. Unlike ARTS, mutant forms of ARTS as well as PNUTL2 did not promote apoptosis in STS treated cells. P-values (Anova) were 0.03 for non-treated cells, compared to 0.000004 for 1 hr and 0.00001 for 3 hr STS treatment.

FIG. 5. ARTS and XIAP co-localize during apoptosis. Immunofluorescence assay of COS-7 cells with anti-ARTS antibody (left column) and anti-XIAP antibody (second column from left). Nuclei are stained with Dapi (third column from left). Merged images are shown in the right column. Cells were untreated (top panel) or treated with 100 mM etoposide for 2 hours (middle panel) or 16 hours (lower panel). Cells were analyzed using confocal microscopy (magnification 60). Non-apoptotic cells had very little overlap between ARTS and XIAP staining (top panel), with ARTS primarily localized to mitochondria and XIAP staining cytoplasmic, with some peri-nuclear concentration. Two hours after induction of apoptosis, co-localization of ARTS and XIAP occurred primarily near the nucleus (middle panel). After 16 hr both proteins perfectly co-localized in the nucleus.

FIG. 6. Upon apoptotic induction, ARTS binds higher levels of XIAP in a caspase-independent manner. A, Co-IP using anti ARTS antibodies was performed on lysates of NRP-154 cells without apoptotic treatment, or treated with 100 μM etoposide for 3 or 6 hours, followed by Western blot analysis with anti XIAP antibodies. B. Co-IP was performed on COS-7 cells without apoptotic treatment, or treated with 1 μM staurosporin for 1 or 3 hour, with or without BOC treatment. C. XIAP levels in the lysates used for IP. During apoptotic induction, both NRP-154 cells and COS-7 cells showed a significant increase in ARTS binding to XIAP. ARTS-XIAP interactions did not depend on either caspase activity or apoptosis.

FIG. 7. ARTS causes down-regulation of XIAP levels during apoptosis. A. COS 7 cells were transiently transfected with either XIAP alone, or co-transfected with XIAP and AU5-ARTS, then treated with or without 100 μM etoposide. IP with anti-myc antibodies was performed, followed by Western blot with anti-XIAP antibody, B. COS-7-pE cells and COS-7-pE cells stably transfected with AU5-ARTS were analyzed with anti-XIAP, anti-AU5, anti-H2A.X and anti-actin antibodies. COS-7 pE-ARTS cells expressing high levels of ARTS exhibited significantly reduced levels of XIAP. The phospho-histone H2A.X was used to identify cells undergoing apoptosis. C. COS-7 cells were transiently transfected with AU5-ARTS or AU5-PNUTL2, treated with 1 mM staurosporin (STS) for 0, 2 or 5 hours, and analyzed by Western blot using anti-XIAP and an anti-ARTS antibody that recognizes the common N′ terminus of ARTS and H5. XIAP levels were significantly reduced in cells transfected with ARTS but not PNUTL2.

### DETAILED DESCRIPTION OF THE INVENTION

The present invention demonstrates that ARTS promotes apoptosis through binding and inhibition of XIAP, providing a novel mechanism for induction of apoptosis. Upon induction of apoptosis, ARTS is released from mitochondria and forms a stable complex with XIAP. Binding of ARTS to XIAP causes a significant reduction in XIAP levels and leads to caspase activation and cell death.

Thus, in one embodiment, the present invention provides a method of identifying a domain or region of an ARTS protein or a variant or homologue thereof that mediates a biological activity, the method comprising the steps of: (a) generating a subset of test fragments of the ARTS protein that bind to an IAP protein or a variant or homologue thereof; and (b) analyzing the subset for inclusion of a polypeptide common to all of the test fragments in the subset, whereby the polypeptide defines a domain or region of an ARTS protein or a variant or homologue thereof that mediates a biological activity.

In one embodiment, the ARTS protein, variant, or homologue thereof of compositions and methods of the present invention has a sequence as set forth in SEQ ID No: 6. In another embodiment, the ARTS protein, variant, or homologue thereof is homologous to the sequence set forth in SEQ ID No: 6:

(SEQ ID No: 6)

MIKRFLEDTTDDGELSKFVKDFSGNASCHPPEAKTWASRPQVPEPRPQAP
DLYDDDLEFRPPSRPQSSDNQQYFCAPAPLSPSARPRSPWGKLDPYDSSE
DDKEYVGFATLPNQVHRKSVKKGFDFTLMVAGESGLGKSTLVNSLFLTDL
YRDRKLLGAEERIMQTVEITKHAVDIEEKGVRLRLTIVDTPGFGDAVNNT
ECWKPVAEYIDQQFEQYFRDESGLNRKNIQDNRVHCCLYFISPFGHGYGP
SLRLLAPPGAVKGTGQEHQGQGCH.

In another embodiment, the ARTS protein is any other ARTS protein known in the art. Each ARTS protein represents a separate embodiment of the present invention.

In one embodiment, the domain or region of an ARTS protein of the present invention is a minimum region of the ARTS protein that is capable of performing the specified biological function. In one embodiment, “minimum region” refers to the smallest portion of the protein that retains the described activity. In another embodiment, “minimum region” refers to the smallest portion of the protein that retains a biological activity similar to that of the full-length protein. In another embodiment, the “minimum region” does not exactly define the smallest portion of the protein that exhibits the property, but rather approximately defines it. In one embodiment, “approximately” refers to within about 10 amino acids. In another embodiment, “approximately” refers to within about 8 amino acids. In another embodiment, “approximately” refers to within about 6 amino acids. In another embodiment, “approximately” refers to within about 4 amino acids. In another embodiment, “approximately” refers to within about 4 amino acids. Each possibility represents a separate embodiment of the present invention.

In one embodiment, “biological activity” refers to an apoptosis. In another embodiment, “biological activity” refers to a killing of a cancer cell. In another embodiment, “biological activity” refers to an activity against a psoriasis. In another embodiment, “biological activity” refers to an activity against a tuberculosis infection. In another embodiment, “biological activity” refers to an activity against a Bartonella infection. In another embodiment, “biological activity” refers to an activity against a vitiligo. In another embodiment, “biological activity” refers to an activity against an atopic dermatitis. In another embodiment, “biological activity” refers to an activity against a hyper-proliferative dermiatosis. In another embodiment, “biological activity” refers to an activity against a UV-responsive dermatosis. In another embodiment, “biological activity” refers to an activity against a lymphohistiocytosis.

In one embodiment, “biological activity” refers to a binding to an IAP protein. In one embodiment, “biological activity” refers to a release of a caspase from an IAP protein. In one embodiment, the IAP protein is an XIAP protein. In another embodiment, the IAP protein is a CIAP protein. In another embodiment, the IAP protein is any other IAP protein known in the art. Each possibility represents a separate embodiment of the present invention.

In one embodiment, the caspase of the present invention is caspase 9. In another embodiment, the caspase is any other caspase known in the art. Each possibility represents a separate embodiment of the present invention.

As described herein, the present invention shows that over-expression of ARTS increases caspase activity, and that ARTS acts downstream of extracellular pro-apoptotic stimuli, but upstream of caspases to promote apoptosis (Examples 1 and 4) ARTS binds to XIAP, and interaction with XIAP correlates with induction of apoptosis (Examples 5 and 7), showing that binding of ARTS to XIAP causes apoptosis In addition, the present invention shows that a region including some or all of the C-terminal 68 amino acids of ARTS (approximately amino acids 207-274), in one embodiment, is responsible for XIAP binding, showing that ARTS fragments can have biological activity.

In another embodiment, a region consisting of approximately the C-terminal 68 amino acids of ARTS mediates XIAP binding. In another embodiment, a region consisting of approximately the C-terminal 70 amino acids mediates XIAP binding. In another embodiment, a region consisting of approximately the C-terminal 75 amino acids mediates XIAP binding. In another embodiment, a region consisting of approximately the C-terminal 80 amino acids mediates XIAP binding. In another embodiment, a region consisting of approximately the C-terminal 85 amino acids mediates XIAP binding. In another embodiment, a region consisting of approximately the C-terminal 90 amino acids mediates XIAP binding. In another embodiment, a region consisting of approximately the C-terminal 95 amino acids mediates XIAP binding. In another embodiment, a region consisting of approximately the C-terminal 100 amino acids mediates XIAP binding. In another embodiment, a region consisting of approximately the C-terminal 110 amino acids mediates XIAP binding. In another embodiment, a region consisting of approximately the C-terminal 65 amino acids mediates XIAP binding. In another embodiment, a region consisting of approximately the C-terminal 60 amino acids mediates XIAP binding. In another embodiment, a region consisting of approximately the C-terminal 55 amino acids mediates XIAP binding. In another embodiment, a region consisting of approximately the C-terminal 50 amino acids mediates XIAP binding. In another embodiment, a region consisting of approximately the C-terminal 45 amino acids mediates XIAP binding. In another embodiment, a region consisting of approximately the C-terminal 40 amino acids mediates XIAP binding. In another embodiment, a region consisting of approximately the C-terminal 35 amino acids mediates XIAP binding. In another embodiment, a region consisting of approximately the C-terminal 30 amino acids mediates XIAP binding.

In another embodiment, a region consisting of approximately amino acids 207-269 mediates XIAP binding. In another embodiment, a region consisting of approximately amino acids 207-264 mediates XIAP binding. In another embodiment, a region consisting of approximately amino acids 207-259 mediates XIAP binding. In another embodiment, a region consisting of approximately amino acids 207-254 mediates XIAP binding. In another embodiment, a region consisting of approximately amino acids 207-249 mediates XIAP binding. In another embodiment, a region consisting of approximately amino acids 207-244 mediates XIAP binding. In another embodiment, a region consisting of approximately amino acids 207-239 mediates XIAP binding. In another embodiment, a region consisting of approximately amino acids 234-229 mediates XIAP binding. In another embodiment, a region consisting of approximately amino acids 207-224 mediates XIAP binding. In another embodiment, a region consisting of approximately amino acids 207-219 mediates XIAP binding. In another embodiment, a region consisting of approximately amino acids 207-216 mediates XIAP binding. Each region of ARTS represents a separate embodiment of the present invention.

In one embodiment, “mediates binding” means that the specified region mediates XIAP binding alone; i.e, without requiring additional region(s) of ARTS. In another embodiment, “mediates binding” means that the specified region mediates binding together with one or more additional regions of ARTS. Each possibility represents a separate embodiment of the present invention.

In one embodiment, the IAP protein of methods and compositions of the present invention is an X-link inhibitor of apoptosis protein (XIAP) or a variant or homologue thereof. In another embodiment, the IAP protein has a sequence as set forth in SEQ ID No: 7. In another embodiment, the IAP protein has a sequence homologous to the sequence set forth in SEQ ID No: 7).

(SEQ ID No: 7)

MTFNSFEGSKTCVPADINKEEEFVEEFNRLKTFANFPSGSPVSASTLARA
GFLYTGEGDTVRCFSCHAAVDRWQYGDSAVGRHRKVSPNCRFINGFYLEN
SATQSTNSGIQNGQYKVENYLGSRDHFALDRPSETHADYLLRTGQVVDIS
DTIYPRNPAMYSEEARLKSFQNWPDYAHLTPRELASAGLYYTGIGDQVQC
FCCGGKLKNWEPCDRAWSEHRRHFPNCFFVLGRNLNIRSESDAVSSDRNF
PNSTNLPRNPSMADYEARIFTFGTWIYSVNKEQLARAGFYALGEGDKVKC
FHCGGGLTDWKPSEDPWEQHAKWYPGCKYLLEQKGQEYINNIHLTHSLEE
CLVRTTEKTPSLTRRIDDTIFQNPMVQEAIRMGFSFKDIKKIMEEKIQIS
GSNYSLEVLVADLVNAQKDSMQDESSQTSLQKEISTEEQLRRLQEEKLCK
ICMDRNIAIVFVPCGHLVTCKQCAEAVDKCPMCYTVITFKQKIFMS.

In another embodiment, the LAP protein is any other IAP protein known in the art. Each IAP protein represents a separate embodiment of the present invention.

In one embodiment, the polypeptide common to all of the test fragments in the subset defines a “common region,” or region or domain of the protein common to all of the test fragments in the subset. The “common region” defines, in one embodiment, the minimal domain necessary for the described activity. In another embodiment, the common region includes the C-terminal 68 amino acids of ARTS. In another embodiment, the common region includes a portion of the C-terminal 68 amino acids of ARTS. Each possibility represents a separate embodiment of the present invention.

In another embodiment, the present invention provides a mimetic compound of the common region identified by one of the methods of identifying a region of an ARTS protein or a variant or homologue thereof that mediates a biological activity.

In one embodiment, the mimetic compound is a non-peptide compound. In another embodiment, the mimetic compound is a peptide compound. In another embodiment, the mimetic compound is any other type of compound known in the art. Each possibility represents a separate embodiment of the present invention.

In another embodiment, the present invention provides a method of inducing an apoptosis, comprising administering the mimetic compound of the present invention. In another embodiment, the present invention provides a method of a killing of a cancer cell, comprising administering the mimetic compound of the present invention. In another embodiment, the present invention provides a method of treating a psoriasis, comprising administering the mimetic compound of the present invention. In another embodiment, the present invention provides a method of treating a tuberculosis infection, comprising administering the mimetic compound of the present invention. In another embodiment, the present invention provides a method of treating a Bartonella infection, comprising administering the mimetic compound of the present invention. In another embodiment, the present invention provides a method of treating a vitiligo, comprising administering the mimetic compound of the present invention. In another embodiment, the present invention provides a method of treating an atopic dermatitis, comprising administering the mimetic compound of the present invention. In another embodiment, the present invention provides a method of treating a hyper-proliferative or UV-responsive dermatosis, comprising administering the mimetic compound of the present invention. In another embodiment, the present invention provides a method of treating a lymphohistiocytosis, comprising administering the mimetic compound of the present invention. In another embodiment, the present invention provides a method of treating any other apoptosis-related disease or disorder, comprising administering the mimetic compound of the present invention. Each disease or disorder represents a separate embodiment of the present invention.

In one embodiment, the cancer cell of the present invention is a breast cancer cell. In another embodiment, the cancer cell is a prostate cancer cell. In another embodiment, the cancer cell is, a head and neck cancer cell. In another embodiment, the cancer cell is an ovarian cancer cell. In another embodiment, the cancer cell is a pancreatic cancer cell. In another embodiment, the cancer cell is a colon cancer cell. In another embodiment, the cancer cell is a glioblastoma cell. In another embodiment, the cancer cell is a cervical cancer cell. In another embodiment, the cancer cell is a lung cancer cell. In another embodiment, the cancer cell is a gastric cancer cell. In another embodiment, the cancer cell is a liposarcoma cell. In another embodiment, the cancer cell is a sarcoma cell. In another embodiment, the cancer cell is a carcinoma cell. In another embodiment, the cancer cell is a lymphoma cell. In another embodiment, the cancer cell is a leukemia cell. In another embodiment, the cancer cell is a myeloma cell. In another embodiment, the cancer cell is a melanoma cell. In another embodiment the cancer cell is any other type of cancer cell known in the art. Each possibility represents a separate embodiment of the present invention.

Methods of assessing apoptosis as well known in the art, and include, e.g., morphological methods (Examples; Jacquel A et al, FASEB J. 2003 November; 17(14):2160-2), DNA fragmentation (Jacquel et al, ibid), expression of apoptosis-associated proteins (ibid), mitochondrial membrane depolarization (ibid), flow cyometry (McEwen A, J Pathol. 2003 November; 201(3):395-403), and Annexin-V and Propidium iodide staining (Liu J et al, Acta Med Okayama. 2003 October; 57(5):209-16). Each assay represents a separate embodiment of the present invention.

In another embodiment, the present invention provides a method of identifying a compound useful for a chemotherapy of a neoplastic disease or disorder, comprising testing the mimetic compound of the present invention for an activity against the neoplastic disease or disorder, whereby, if the mimetic compound exhibits an activity against the neoplastic disease or disorder, then the mimetic compound is a compound useful for a chemotherapy of a neoplastic disease or disorder.

In one embodiment, the neoplastic disease or disorder is a cancer, a psoriasis, a vitiligo, an atopic dermatitis, a hyper-proliferative or UV-responsive dermatosis, or a lymphohistiocytosis.

In another embodiment, the neoplastic disease or disorder is a breast cancer, a prostate cancer, a head and neck cancer, an ovarian cancer, a pancreatic cancer, a colon cancer, a glioblastoma, a cervical cancer, a lung cancer, a gastric cancer, a liposarcoma, a sarcoma, a carcinoma, a lymphoma, a leukemia, a myeloma, or a melanoma.

In another embodiment, the neoplastic disease or disorder comprises a decreased level of the ARTS protein, variant, or homologue thereof. In another embodiment, the neoplastic disease or disorder comprises a decreased function of the ARTS protein, variant, or homologue thereof. In another embodiment, the neoplastic disease or disorder comprises a decreased function of any ARTS protein known in the art. In one embodiment, the decreased level or function is an undetectable level or function. In another embodiment, the decreased level or function is a detectable but reduced level or function. In another embodiment, the decreased function is any function of the ARTS protein known in the art. Each possibility represents a separate embodiment of the present invention.

In one embodiment, the decreased level or function is a result of a mutation in a coding sequence of the ARTS protein, variant, or homologue thereof. In another embodiment, the decreased level or function is a result of a modification in a coding sequence of the ARTS protein, variant, or homologue thereof. In another embodiment, the decreased level or function is a result of a mutation in a regulatory sequence of the ARTS protein, variant, or homologue thereof. In another embodiment, the decreased level or function is a result of a modification in a regulatory sequence of the ARTS protein, variant, or homologue thereof. In one embodiment, the mutation or modification in a regulatory sequence is in a promoter of the ARTS protein, variant, or homologue thereof. In one embodiment, the mutation or modification in a regulatory sequence is in an enhancer of the ARTS protein, variant, or homologue thereof. In another embodiment, the modification is a methylation. In another embodiment, the mutation or modification results in a decrease of an amount or concentration of the ARTS protein in cells of the neoplastic disease or disorder. Each possibility represents a separate embodiment of the present invention.

In another embodiment, the decreased level or function is a result of a mutation or alteration in the cell that increases degradation of the ARTS protein, variant, or homologue thereof. In one embodiment, the degradation is due to a ubiquitination. In another embodiment, the degradation is due to another cellular method of protein degradation. Each possibility represents a separate embodiment of the present invention.

In another embodiment, the neoplastic disease or disorder comprises an increased level of an IAP protein, variant, or homologue thereof. In another embodiment, the neoplastic disease or disorder comprises an increased function of an IAP protein, variant, or homologue thereof. In one embodiment, administration of an ARTS protein, or mimetic or derivative thereof compensates for the increased level or function of an IAP protein, restoring the capability of apoptosis to the cell. Each possibility represents a separate embodiment of the present invention.

In another embodiment, the present invention provides a method of identifying a domain or region of an ARTS protein or a variant or homologue thereof that mediates a biological activity, the method comprising the steps of: (a) generating a subset of test fragments of the ARTS protein, variant, or homologue thereof that reduce a level of an IAP protein or a variant or homologue thereof; and (b) analyzing the subset for inclusion of a polypeptide common to all of the test fragments in the subset, whereby the polypeptide defines a domain or region of an ARTS protein or a variant or homologue thereof that mediates a biological activity. As demonstrated herein, ARTS reduces XIAP levels, causing apoptosis (Example 10).

In another embodiment, the present invention provides a method of identifying a domain or region of an ARTS protein or a variant or homologue thereof that mediates a biological activity, the method comprising the steps of: (a) generating a subset of test fragments of the ARTS protein that induce the translocation of an IAP protein, variant, or homologue thereof; and (b) analyzing the subset for inclusion of a polypeptide common to all of the test fragments in the subset, whereby the polypeptide defines a domain or region of an ARTS protein or a variant or homologue thereof that mediates a biological activity. The present invention shows that ARTS/XIAP complexes are translocated to the nucleus during apoptosis (Examples 2 and 8, showing that translocation of XIAP to the nucleus has biological relevance and plays a role in apoptosis.

In another embodiment, the present invention provides a method of inducing apoptosis in a cell, comprising the step of inhibiting or reducing a degradation of an ARTS protein or a variant or homologue thereof. Example 3 of the present invention shows that ARTS is rapidly degraded in non-apoptotic cells. Thus, inhibiting such degradation results in rapid accumulation of ARTS, and stimulates apoptosis, as shown in the present invention.

In one embodiment, the degradation is mediated by a proteasome. In another embodiment, the degradation is mediated by any other cellular protease. Each possibility represents a separate embodiment of the present invention.

In another embodiment, the present invention provides an isolated complex, comprising an ARTS protein or a variant or homologue thereof and an IAP protein or a variant or homologue thereof. The existence of ARTS-XIAP complexes was shown by several biochemical methods (Example 5), and immuno-fluorescence (Example 8)

In another embodiment, the present invention provides a compound that selectively binds to the isolated complex of the present invention.

In another embodiment, the present invention provides a compound that inhibits a formation of the isolated complex of the present invention.

In another embodiment, the present invention provides a compound that disrupts the isolated complex of the present invention.

In one embodiment, the compound disrupts or inhibits formation of the complex by sterically blocking interaction between ARTS and the IAP protein. In another embodiment, the compound disrupts or inhibits formation of the complex by sterically blocking interaction between ARTS and another component of the complex. In another embodiment, the compound disrupts or inhibits formation of the complex by sterically blocking interaction between the IAP protein and another component of the complex. In another embodiment, the compound disrupts or inhibits formation of the complex by interacting with ARTS protein alone, for example by inducing a conformation change that precludes or reduces interation with the IAP protein or another component of the complex. In another embodiment, the compound disrupts or inhibits formation of the complex by interacting with the IAP protein alone, precluding or reducing interation with or another component of the complex. Each possibility represents a separate embodiment of the present invention.

In another embodiment, the present invention provides a method of testing a compound for an ability to inhibit or reduce an incidence of an apoptosis, comprising determining whether the compound disrupts the isolated complex of the present invention, whereby if the compound disrupts the isolated complex of the present invention, then the compound inhibits or reduces an incidence of an apoptosis.

In another embodiment, the present invention provides a method of treating or reducing an incidence of a neuro-degenerative disease, comprising administering a compound that disrupts the isolated complex of the present invention, thereby treating or reducing an incidence of a neuro-degenerative disease.

In another embodiment, the present invention provides a method of treating or reducing an incidence of an ischemic injury, comprising administering a compound that disrupts the isolated complex of the present invention, thereby treating or reducing an incidence of an ischemic injury.

In another embodiment, the present invention provides a method of treating or reducing an incidence of a myelodysplasia, comprising administering a compound that disrupts the isolated complex of the present invention, thereby treating or reducing an incidence of a myelodysplasia.

In another embodiment, the present invention provides a method of treating or reducing an incidence of an atherosclerosis, comprising administering a compound that disrupts the isolated complex of the present invention, thereby treating or reducing an incidence of an atherosclerosis.

In another embodiment, the present invention provides a method of treating or reducing an incidence of an auto-immune disease, comprising administering a compound that disrupts the isolated complex of the present invention, thereby treating or reducing an incidence of an auto-immune disease.

In another embodiment, the present invention provides a method of treating or reducing an incidence of a Crohn's disease or ulcerative colitis, comprising administering a compound that disrupts the isolated complex of the present invention, thereby treating or reducing an incidence of a Crohn's disease or ulcerative colitis.

In another embodiment, the present invention provides a method of treating or reducing an incidence of a cytopenia, comprising administering a compound that disrupts the isolated complex of the present invention, thereby treating or reducing an incidence of a cytopenia

In another embodiment, the present invention provides a method of treating or reducing an incidence of a pancreatitis, comprising administering a compound that disrupts the isolated complex of the present invention, thereby treating or reducing an incidence of a pancreatitis.

In another embodiment, the present invention provides a method of treating or reducing an incidence of a periodontitis, comprising administering a compound that disrupts the isolated complex of the present invention, thereby treating or reducing an incidence of a periodontitis.

In another embodiment, the present invention provides a method of testing a compound for an ability to inhibit or reduce an incidence of an apoptosis, comprising determining whether the compound prevents a binding of an ARTS protein or a variant or homologue thereof to an XIAP protein or a variant or homologue thereof, whereby if the compound prevents binding of the ARTS protein, variant or homologue thereof, to the IAP protein, variant, or homologue thereof, then the compound inhibits or reduces an incidence of an apoptosis.

In another embodiment, the present invention provides a method of treating or reducing an incidence of a neuro-degenerative disease, comprising administering a compound that prevents a binding of an ARTS protein or a variant or homologue thereof to an XIAP protein or a variant or homologue thereof, thereby treating or reducing an incidence of a neuro-degenerative disease.

In another embodiment, the present invention provides a method of treating or reducing an incidence of an ischemic injury, comprising administering a compound that prevents a binding of an ARTS protein or a variant or homologue thereof to an XIAP protein or a variant or homologue thereof, thereby treating or reducing an incidence of an ischemic injury.

In another embodiment, the present invention provides a method of treating or reducing an incidence of a myelodysplasia, comprising administering a compound that prevents a binding of an ARTS protein or a variant or homologue thereof to an XIAP protein or a variant or homologue thereof, thereby treating or reducing an incidence of a myelodysplasia.

In another embodiment, the present invention provides a method of treating or reducing an incidence of an atherosclerosis, comprising administering a compound that prevents a binding of an ARTS protein or a variant or homologue thereof to an XIAP protein or a variant or homologue thereof, thereby treating or reducing an incidence of an atherosclerosis.

In another embodiment, the present invention provides a method of treating or reducing an incidence of an auto-immune disease, comprising administering a compound that prevents a binding of an ARTS protein or a variant or homologue thereof to an XIAP protein or a variant or homologue thereof, thereby treating or reducing an incidence of an auto-immune disease.

In another embodiment, the present invention provides a method of treating or reducing an incidence of a Crohn's disease or ulcerative colitis, comprising administering a compound that prevents a binding of an ARTS protein or a variant or homologue thereof to an XIAP protein or a variant or homologue thereof, thereby treating or reducing an incidence of a Crohn's disease or ulcerative colitis.

In another embodiment, the present invention provides a method of treating or reducing an incidence of a cytopenia, comprising administering a compound that prevents a binding of an ARTS protein or a variant or homologue thereof to an XIAP protein or a variant or homologue thereof, thereby treating or reducing an incidence of a cytopenia.

In another embodiment, the present invention provides a method of treating or reducing an incidence of a pancreatitis, comprising administering a compound that prevents a binding of an ARTS protein or a variant or homologue thereof to an XIAP protein or a variant or homologue thereof, thereby treating or reducing an incidence of a pancreatitis.

In another embodiment, the present invention provides a method of treating or reducing an incidence of a periodontitis, comprising administering a compound that prevents a binding of an ARTS protein or a variant or homologue thereof to an XIAP protein or a variant or homologue thereof, thereby treating or reducing an incidence of a periodontitis.

In one embodiment, the neuro-degenerative disease is a Huntington's disease. In another embodiment, the neuro-degenerative disease is an amyotrophic lateral sclerosis. In another embodiment, the neuro-degenerative disease is an Alzheimer's disease. In another embodiment, the neuro-degenerative disease is a Parkinson's disease. In another embodiment, the neuro-degenerative disease is a Niemann-Pick disease. In another embodiment, the neuro-degenerative disease is any other neuro-degenerative disease known in the art.

In one embodiment, the cytopenia is a lymphopenia. In another embodiment, the cytopenia is an anemia. In another embodiment, the cytopenia is a leukopenia. In another embodiment, the cytopenia is a neutropenia. In another embodiment, the cytopenia is a thrombocytopenia. In another embodiment, the cytopenia is a pancytopenia. In another embodiment, the cytopenia is any other type of cytopenia known in the art.

Each type of each of the above diseases and disorders represents a separate embodiment of the present invention.

In one embodiment, a subset of fragments of the ARTS protein that bind to the IAP protein is generated by: (a) generating a test fragment of the ARTS protein; (b) testing a binding of the test fragment to the IAP protein or a variant or homologue thereof; and (c) repeating steps (a)-(b) to generate the subset of fragments of ARTS protein that bind to IAP, variant, or homologue thereof.

In one embodiment, a subset of fragments of the ARTS protein that bind to the IAP protein is generated by: (a) generating a test fragment of the ARTS protein; (b) testing a binding of the test fragment to the IAP protein or a variant or homologue thereof; and (c) repeating steps (a)-(b) to generate the subset of fragments of ARTS protein that bind to LAP, variant, or homologue thereof.

In another embodiment, a subset of ARTS fragments that reduce a level of an IAP protein, variant, or homologue thereof is generated by (a) generating a test fragment of the ARTS protein, variant, or homologue thereof; (b) contacting a cell with the test fragment; (c) testing an ability of the test fragment to reduce a level of an IAP protein or a variant or homologue thereof in the cell; and (d) repeating steps (a)-(b) to generate the subset of fragments of ARTS protein, variant, or homologue thereof that reduce the level of an IAP protein, variant, or homologue thereof.

In another embodiment, a subset of ARTS fragments that induce a translocation of an IAP protein, variant, or homologue thereof is generated by (a) generating a test fragment of the ARTS protein, variant, or homologue thereof, (b) contacting a cell with the test fragment; (c) testing an ability of the test fragment to induce a translocation of an IAP protein, variant, or homologue thereof in the cell; and (d) repeating steps (a)-(b) to generate the subset of fragments of ARTS protein, variant, or homologue thereof that induce a translocation of an IAP protein, variant, or homologue thereof.

ARTS was shown in the present invention to increase apoptosis in some cell types even in the absence of pro-apoptotic stimuli. The basis for the differential effect in different cell lines of expression of ARTS in the absence of pro-apoptotic stimuli on caspase-3 activity is likely to be the fact that HL-60 cells are devoid of endogenous ARTS, whereas all the other cell lines express various levels of endogenous ARTS protein. It was found that variations in expression levels of transfected ARTS are responsible for the observed differential response of cells: higher expression levels of ARTS lead to increased apoptosis in the absence of pro-apoptotic stimuli

Thus, in one embodiment, the ARTS fragment or mimetic compound of the present invention induces apoptosis or treats one of the indicated conditions in the absence of additional pro-apoptotic stimuli. In another embodiment, the ARTS fragment or mimetic compound induces apoptosis or treats one of the indicated conditions in combination with pro-apoptotic stimuli. Each possibility represents a separate embodiment of the present invention.

The findings of the present invention show that ARTS can interact with multiple IAPs, since inactivation of XIAP alone cannot account for the induction of apoptosis. Thus, in another embodiment, the IAP protein in compositions and methods of the present invention is CIAP. CIAP has been shown in the present invention to interact with ARTS. In another embodiment, the IAP protein is any other member of the IAP family. Each possibility represents a separate embodiment of the present invention.

As shown in the present invention, binding of ARTS to an IAP protein does not require caspase activity and the execution of cell death. On the other hand, caspase inhibitors blocked the nuclear translocation of ARTS. Taken together, these results indicate the following role of ARTS for the induction of apoptosis (FIG. 8): In living cells, ARTS is localized to mitochondria. Upon receiving an apoptotic stimulus, ARTS is released from mitochondria by a caspase-independent mechanism and binds an IAP protein, reducing levels of an IAP protein. In one embodiment, levels of an IAP protein are reduced by proteasome-mediated degradation. In another embodiment, levels of an IAP protein are reduced by increased auto-ubiquitination of an IAP protein. In another embodiment, levels of an IAP protein are reduced by translocation of ARTS-LIP protein complexes to the nucleus. Each mechanism of reducing levels of an IAP protein represents a separate embodiment of the present invention.

In another embodiment, as a result of down-regulation of levels of an IAP protein, caspase activity becomes de-repressed, and apoptosis is facilitated. The release of Smac/Diablo from mitochondria requires, in one embodiment, caspase activity. Therefore, in one embodiment, ARTS acts at an earlier stage of apoptosis than Smac/Diablo,

In another embodiment, ARTS acts at an earlier stage of apoptosis than one or more caspase proteins. In another embodiment, ARTS acts at an earlier stage of apoptosis than all caspase proteins participating in apoptosis. In one embodiment, the relatively early placement of ARTS in the apoptosis process allow ARTS and mimetics and derivatives thereof to function in apoptotic or other processes that do not require functional caspase activity. In another embodiment, the relatively early placement of ARTS in the apoptosis process allow ARTS and mimetics and derivatives thereof to function in response to a wide variety of apoptotic stimuli. In another embodiment, the relatively early placement of ARTS in the apoptosis process allow ARTS and mimetics and derivatives thereof to function in a wide variety of apoptotic processes. In another embodiment, the relatively early placement of ARTS in the apoptosis process allow ARTS and mimetics and derivatives thereofto function in response to a wide variety of cell types. Each possibility represents a separate embodiment of the present invention.

In one embodiment, ARTS promotes the assembly of multi-protein complexes between IAPs and other cell death regulators. In one embodiment, the complexes include IAP antagonists. In another embodiment, the complexes include ubiquitin pathway proteins. In another embodiment, ARTS employs novel protein motifs to carry out its IAP-inhibiting activities. Each possibility represents a separate embodiment of the present invention.

The terms “homology,” “homologous,” etc, when in reference to any protein or peptide, refer in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

Homology is, in one embodiment, determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, homology is determined is via determination of candidate sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In one embodiment, the subset of ARTS fragments is generated by deletion mutagenesis. Methods of deletion mutagenesis are well known in the art, and are described, for example in (Henikoff S, Gene 28(3): 351-9, 1984; and Pues H et al, Nucleic Acids Research, 25 (6): 1303-1304, 1997). Each method of deletion mutagenesis represents a separate embodiment of the present invention.

In other embodiments, a method of the present invention treats any disease, disorder, or symptom associated with an abnormally high degree or amount of apoptosis. In other embodiments, a method of the present invention treats any disease, disorder, or symptom associated with an abnormally low degree or amount apoptosis. In another embodiment, a method of the present invention treats any disease, disorder, or symptom associated with a temporally abnormal pattern of apoptosis. In another embodiment, a method of the present invention treats any disease, disorder, or symptom associated with a spatially abnormal pattern of apoptosis. Each possibility represents a separate embodiment of the present invention.

Pharmaceutical Compositions

As contemplated herein, the present invention relates to the use of an apoptosis-modifying compound and/or its analog, derivative, isomer, metabolite, pharmaceutically acceptable salt, pharmaceutical product, hydrate, N-oxide, or combinations thereof for treating, preventing, suppressing, inhibiting or reducing the incidence of an apoptosis-mediated disorder. Thus, in one embodiment, the methods of the present invention comprise administering an analog of the apoptosis-modifying compound. In another embodiment, the methods of the present invention comprise administering a derivative of the apoptosis-modifying compound. In another embodiment, the methods of the present invention comprise administering an isomer of the apoptosis-modifying compound. In another embodiment, the methods of the present invention comprise administering a metabolite of the apoptosis-modifying compound. In another embodiment, the methods of the present invention comprise administering a pharmaceutically acceptable salt of the apoptosis-modifying compound. In another embodiment, the methods of the present invention comprise administering a pharmaceutical product of the apoptosis-modifying compound. In another embodiment, the methods of the present invention comprise administering a hydrate of the apoptosis-modifying compound. In another embodiment, the methods of the present invention comprise administering an N-oxide of the apoptosis-modifying compound. In another embodiment, the methods of the present invention comprise administering any of a combination of an analog, derivative, isomer, metabolite, pharmaceutically acceptable salt, pharmaceutical product, hydrate or N-oxide of the apoptosis-modifying compound.

As used herein, “pharmaceutical composition” means a “therapeutically effective amount” of the active ingredient, i.e. the apoptosis-modifying compound, together with a pharmaceutically acceptable carrier or diluent. A “therapeutically effective amount” refers, in one embodiment, to that amount which provides a therapeutic effect for a given condition and administration regimen.

The pharmaceutical compositions containing the apoptosis-modifying compound can be administered to a subject by any method known to a person skilled in the art, such as parenterally, paracancerally, trans-mucosally, trans-dermally, intramuscularly, intravenously, intra-dermally, subcutaneously, intra-peritonealy, intra-ventricularly, intra-cranially, intra-vaginally or intra-tumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In one embodiment of the present invention, the apoptosis-modifying compounds are formulated in a capsule. In accordance with this embodiment, the compositions of the present invention comprise, in addition to the apoptosis-modifying compound active compound and the inert carrier or diluent, a hard gelating capsule.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intraarterially and are thus formulated in a form suitable for intraarterial administration. In another embodiment, the pharmaceutical compositions are administered intramuscularly and are thus formulated in a form suitable for intramuscular administration.

Further, in another embodiment, the pharmaceutical compositions are administered topically to body surfaces and are thus formulated in a form suitable for topical administration. Suitable topical formulations include gels, ointments, creams, lotions, drops and the like. For topical administration, the apoptosis-modifying compound agents or their physiologically tolerated derivatives such as salts, esters, N-oxides, and the like are prepared and applied as solutions, suspensions, or emulsions in a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of apoptosis-modifying compound agent over a period of time.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp 317-327; see generally ibid).

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with of without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymetlhacrylates) and/or adjuvants.

In one embodiment, the pharmaceutical compositions provided herein are controlled-release compositions, i.e. compositions in which the apoptosis-modifying compound is released over a period of time after administration. Controlled- or sustained-release compositions include formulation in lipophilic depots (e.g. fatty acids, waxes, oils). In another embodiment, the composition is an immediate-release composition, i.e. a composition in which all of the apoptosis-modifying compound is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled-release systems are discussed in the review by Langer (Science 249:1527-1533 (1990).

The compositions may also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions that contain an active component, for example by mixing, granulating, or tablet-forming processes, is well understood in the art. The active therapeutic ingredient is often mixed with excipients that are pharmaceutically acceptable and compatible with the active ingredient. For oral administration, the apoptosis-modifying compound agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the apoptosis-modifying compound agents or their physiologically tolerated derivatives such as salts, esters, N-oxides, and the like are converted into a solution, suspension, or emulsion, if desired with the substances customary and suitable for this purpose, for example, solubilizers or other substances.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of the apoptosis-modifying compounds are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts, which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

The term “contacting” means, in one embodiment, that the apoptosis-modifying compound of the present invention is introduced into a sample containing the enzyme in a test tube, flask, tissue culture, chip, array, plate, microplate, capillary, or the like, and incubated at a temperature and time sufficient to permit binding of the apoptosis-modifying compound to the enzyme. Methods for contacting the samples with the apoptosis-modifying compound or other specific binding components are known to those skilled in the art and may be selected depending on the type of assay protocol to be run. Incubation methods are also standard and are known to those skilled in the art.

In another embodiment, the term “contacting” means that the apoptosis-modifying compound of the present invention is introduced into a subject receiving treatment.

In one embodiment, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing a subject in contact with an apoptosis-modifying compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, the methods of the present invention comprise administering an apoptosis-modifying compound as the sole active ingredient. In another embodiment, the present invention provides methods that comprise administering the apoptosis-modifying compounds in combination with one or more therapeutic agents.

### EXPERIMENTAL DETAILS SECTION

### Example 1

### Over-Expression of ARTS Increases Caspase Activity in a Variety of Cell Types

### Materials and Experimental Methods

Mammalian Cell Culture and Plasmids

K562 and HL-60 cells were grown in RPMI 1640 medium. A549, COS-7 cells were grown in Dulbecco's modified Eagle medium (DMEM) with 4.5 grams/liter (g/L) D-glucose. All cells were grown at 37° C. in a 5% CO₂ atmosphere. All media were supplemented with 10% fetal calf serum (FCS), penicillin (100 U/ml), streptomycin (100 micrograms (μg)/ml) and glutamine (2 millimolar [mM]) (Biological Industries, Israel).

pEF1-AU5 and pEF1-AU5-ARTS constructs were used for all ARTS transient transfection experiments. An AU5 tag was attached to the N′ terminus of ARTS as described in (Larisch S et al, Nat Cell Biol 2: 915-921, 2000). The AU5-ARTSmGTP construct was generated by replacing three amino acids (G K S with E N P) at the GTP binding site, using site-directed mutagenesis (QuickChange™, Stratagene) with the primer: GGAGAGTCTGGCCTGGAGAATCCCACACTTGTCAATAGCC (SEQ ID No: 1).

The AU5-ARTS ΔC construct lacks 68 amino acids at the unique ARTS C′-terminal sequence and was generated using PCR with the following primers:

L1:
BamHI-ATCAAGCGTTTCCTGGAGGACACCACGG; (SEQ ID No: 2)
and
S-207:
EcoRI - CTATGCCACAGGCTTCCAGCACTC. (SEQ ID No: 3)

pEF1-AU5-H5, pEF1-AU5-PNTUL2 isoform 3, AU5-ARTSmGTP, AU5-H5 ΔC and AU5-ARTS ΔC were obtained from Dr. Seong-Jin Kim, NCI/NIH.

Transfections

COS-7, K562 and HL-60 cells were transiently transfected using electroporation (Easyject™ plus—Equibio). A549 cells were transiently transfected using lipofectamin™ (Invitrogen) according to the manufacturer's protocol.

Apoptosis Inducers

The following apoptotic agents were used: TGF-beta (10 ng/ml) for 24 hours in medium containing 1% FCS; 100 mg/ml etoposide for 2 or 16 hours (Sigma); staurosporine (STS) (1 mM) for 0-3 hours (Sigma); and arabinoside-c (ara-C, cytosar) (Pharmacia) at 100 mM for 4 hours. In the case of transiently transfected cells, agents were added forty hours after transfections.

Caspase 3 Activity Assays

Caspase 3 activity was tested in K562, HL-60 and A549 cells using the Caspase 3 activity assay kit (Roche) according to the manufacturer's protocol. Caspase 3 activity was tested in COS-7 cells by immunofluorescence staining with anti-active caspase 3 antibodies 1:4000 (R&D systems). Results are presented as fold increase relative to results in cells transfected with control vector without treatment with apoptotic inducers.

Statistical Analysis

P-value (Anova) of K562 and HL-60 cells were 0.02 and 0.001 respectively; A549 p-value (Anova) was 0.09.

### Results

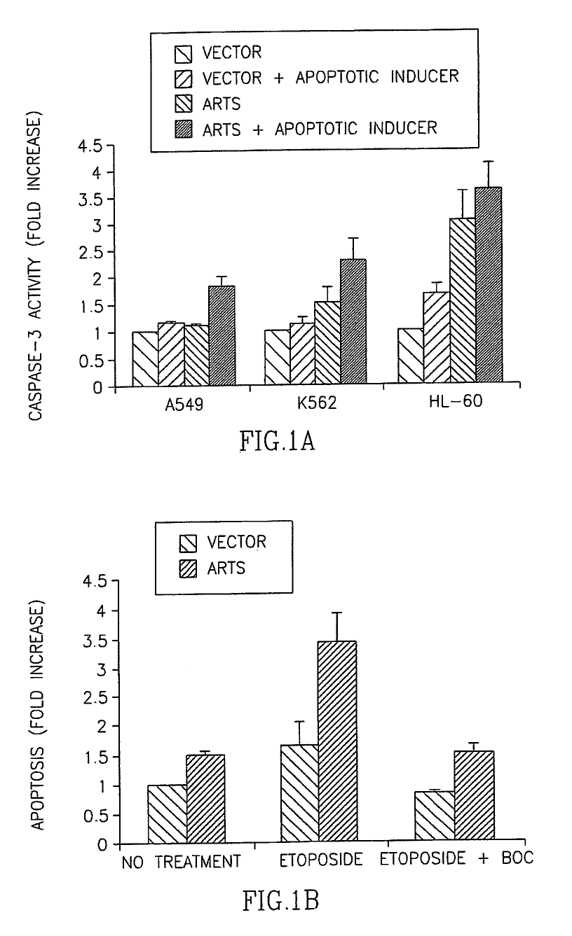

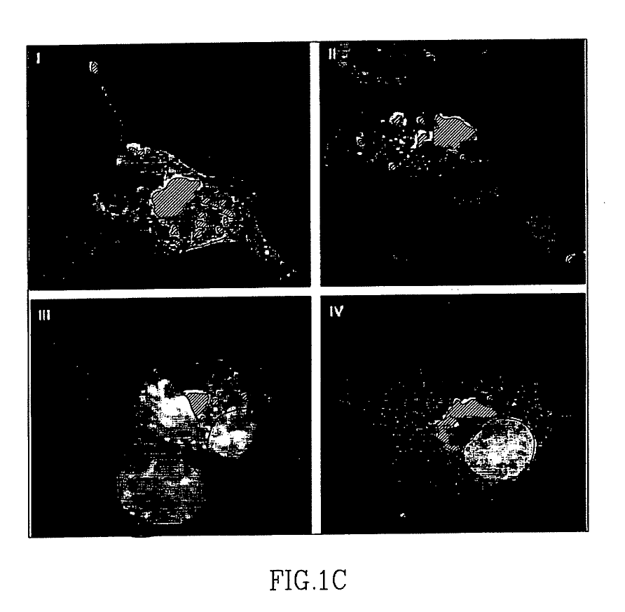

TGF-β and other pro-apoptotic stimuli induce apoptosis via induction of caspase-3 activity. The effect of ARTS on caspase-3 activation in response to TGF-β treatment was examined in a number of cell types and with a number of pro-apoptotic stimuli. The cell types used were two leukemic cell lines (HL-60 and K562), a lung carcinoma cell line (A549), non-tumorigenic rat epithelial (NRP154) cells, and transformed African Green Monkey kidney fibroblast (COS-7) cells. All cell lines were transiently transfected with an-ARTS-expressing vector or an empty vector (negative control). The cells were then exposed to the following pro-apoptotic stimuli: HL-60 and K562 cells were treated with ara-C, A549 cells with TGF-β, and COS-7 cells with etoposide. In all these cases, over-expression of ARTS led to increased caspase-3 activity in response to pro-apoptotic stimuli (FIG. 1A). By contrast, expression of ARTS in the absence of pro-apoptotic stimuli increased caspase-3 activity in HL-60 cells, but had little or no effect on caspase-3 activity in A549, K562 and COS-7 cells.

These findings demonstrate that ARTS protein mediates caspase-3 activation in response to pro-apoptotic stimuli. This function of ARTS is seen in a variety of cell types, and in response to a variety of pro-apoptotic stimuli.

### Example 2

### Caspase Activity Causes ARTS to Translocate from Mitochondria to the Nucleus

### Experimental Methods

Immuno-fluorescence

Cells were fixed with 4% paraformaldehyde in PBS for 20 min at room temperature, washed with PBS, permeabilized with 0.5% Triton-X in PBS for 5 min, and incubated with primary antibodies for 2 hours at room temperature (rabbit polyclonal anti-ARTS (1:20,000; Sigma), monoclonal anti-ARTS, and/or monoclonal anti-XIAP (1:500; used in subsequent Examples). Cells were washed three times with PBS/0.1% Triton-X and incubated with FITC conjugated anti-mouse and Rhodamin^{TX}-conjugated anti-rabbit secondary antibodies (Jackson). Images were analyzed using a confocal laser microscope (Zeiss LSM 510). Mitochondria were visualized with MitoTracker® Red CMXRos (Molecular Probes/Invitrogen).

Caspase Inhibition

For caspase inhibition, we added 40 mM of BOC-Asp(Ome)CH2F (Enzyme Systems Products) one hour prior to addition of the apoptotic agents.

### Results

Next, the role of caspase activity on the sub-cellular distribution of ARTS protein was examined, ARTS translocates from mitochondria to the nucleus during apoptosis. Inhibition of caspases in COS-7 cells transfected with ARTS blocked etoposide-induced apoptosis (FIG. 1B). Using immunofluorescence, the effect of caspase inhibitors on ARTS protein localization was examined in COS-7 cells. Transiently transfected cells were treated with etoposide and incubated with or without caspase inhibitors. In the absence of caspase inhibitors, etoposide-treated cells underwent the typical morphological changes associated with apoptosis and accumulated ARTS in the nucleus (FIG. 1C, panels I and II). Clusters of ARTS-positive mitochondria were observed in the immediate proximity of the nucleus preceding the nuclear entry of ARTS (mitochondrial border shown by a thin white line; merged mitochondria-ARTS fluorescence shown by cross-hatching). The addition of caspase inhibitors blocked the nuclear translocation of ARTS, although the peri-nuclear clustering of ARTS-positive mitochondria was still observed (FIG. 1C, panels III and IV).

The findings of this Example show that caspase activity causes ARTS protein to translocate from mitochondria to the nucleus.

### Example 3

### ARTS Protein Constitutively Leaks from Mitochondria into the Cytoplasm, but is Rapidly Degraded Under Non-Apoptotic Conditions

### Materials and Experimental Methods

Cell Fractionation

Cells were homogenized in 20 mM HEPES-KOH, pH 7.5, 10 mM KCl, 1.5 mM MgCl₂, 1 mM sodium EDTA, 1 mM sodium EGTA, and 1 mM dithiothreitol in the presence of 250 mM sucrose and protease inhibitors. Homogenates were centrifuged at 500×g for 5 min at 4° C., and the supernatant was centrifuged at 10,000×g for 20 min to obtain mitochondria. The pellet was washed and solubilized in TNC buffer (10 mM Tris acetate, pH 8.0, 0.5% Nonidet P-40 (NP-40), 5 mM CaCl₂) with protease inhibitors. Protein concentration was determined with a Micro-BCA kit (Pierce). Anti-OxPhosComplex IV subunit IV antibodies™ (COX IV, Molecular Probes) were used to confirm the identity of the mitochondrial sub-cellular fraction.

### Results

To further confirm the release of ARTS from mitochondria upon pro-apoptotic stimuli, ARTS levels were measured in sub-cellular fractions from both ARTS-transfected and non-transfected cells. Under non-apoptotic conditions, ARTS was mainly detected in mitochondria, though low levels of ARTS were also found in the cytosol (FIG. 1D). When treated with staurosporine (STS) for 1 hour, ARTS levels in the cytosol were strongly increased (FIG. 1D), and this occurred prior to any detectable release of cytochrome c (FIG. 1D, upper panel). ARTS levels decreased following longer exposure to STS, due to the degradation of ARTS in this compartment.

The findings of this Example demonstrate that some ARTS protein constantly leaks from mitochondria into the cytoplasm. However, because ARTS is a very short-lived protein, it cannot accumulate to levels sufficient for the induction of apoptosis under these conditions. Thus, inhibiting degradation of ARTS is a viable strategy for inducing apoptosis.

### Example 4

### The Drosophila Homologue of ARTS Acts Downstream of Reaper, Hid and Grim, but Upstream of Apaf-1

### Experimental Methods

Ectopic expression of Reaper, Hid and Grim under the control of the eye-specific GMR promoter induces apoptosis in cells of the developing retina, resulting in rough and reduced compound eyes. This provides a sensitive assay for mutations in other cell death genes (Bergmann A et al, Oncogene 17: 3215-23, 1998).

### Results

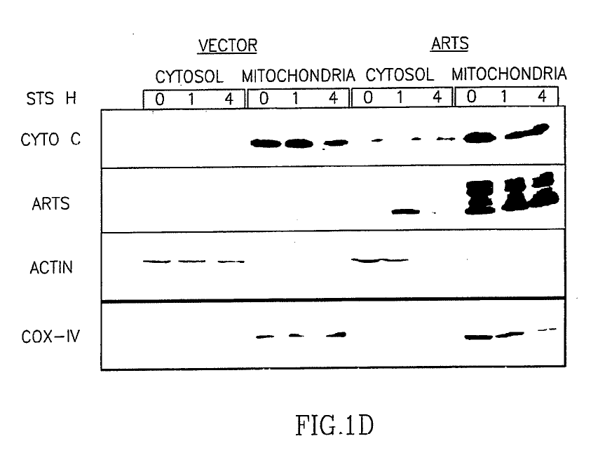

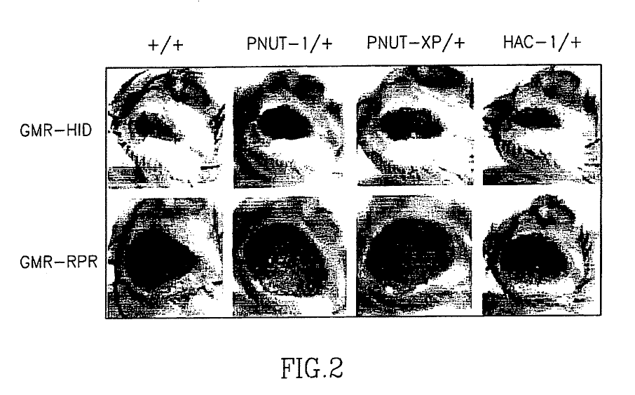

A Drosophila homologue of ARTS in order to gain insight into the point in the apoptotic pathway ARTS in which ARTS acts. The peanut gene of Drosophila is highly homologous to the mammalian Sept4 locus, which encodes both ARTS and H5 (Larisch et al, Nat Cell Biol 2: 915-921, 2000; Neufeld T P et al, Cell 77: 371-379, 1994). In order to examine this possibility, loss-of-function peanut mutations were introduced into Drosophila strains, and their effect on cell killing induced by Reaper, Hid and Grim was assessed. In Drosophila, the IAP antagonists Reaper, Hid and Grim are essential for the induction of virtually all apoptotic cell death (see Experimental Methods section). In animals heterozygous for peanut (pnut1/+ and pnutXP/+), the eye ablation phenotypes induced by Reaper and Hid were significantly reduced (FIG. 2). Similar results were obtained for GMR-Grim. showing that peanut acts downstream of Reaper, Hid and Grim. The extent of suppression by peanut was greater than the consequence of reducing the dosage of the Drosophila Apaf-1 homologue hac-1 (FIG. 2, rightmost panel), indicating that peanut acts upstream of Apaf-1, Apaf-1 is a component of a mitochondrial apoptosis pathway known as the apoptosome that activates caspases. Thus, the ARTS homologue peanut functions downstream of Reaper, Hid and Grim but proximal to the conversion with the Apaf-1 activated caspase pathway.

### Example 5

### ARTS Interacts with XIAP Protein

### Methods

Constructs

The mammalian expression construct encoding Myc epitope-tagged wild-type XIAP in pcDNA3 and the pGEX-XIAP were obtained from Dr. Colin S. Duckett.

For the glutathione-S-transferase (GST)-pull down assays, ARTS was subcloned into the pGEX 4T™ (Pharmacia Biotech) construct; GST-ARTS fusion protein was generated by Polymerase Chain Reaction (PCR) using the following primers:
BamHI-5′-TCGAGGATCCATCAAGCGTTTCCTGGAGGACACCACGG-3′ (SEQ ID No: 4) and
EcoRI-5′ CTAGTGGCAGCCCTGCCCCTGGTGC-3′ (SEQ ID No: 5)
   and cloned into BamHI and EcoRI sites in pGEX 4T.
   Preparation of Beads

For in vitro binding studies, recombinant GST-ARTS or GST-XIAP fusion proteins were purified from bacteria. After sonication, 0.1% Triton X-100 and protease inhibitors (Mini-Complete™, Roche) were added to the bacterial extract followed by 10,000 rotations per minute (RPM) centrifugation. Supernatants of bacterial extracts were collected and incubated in the presence of glutathione-Sepharose 4B beads (Amersham Biosciences) for 30 minute at 4° C. The beads were washed three times.

GST Pull Down Experiments

Cells were lysed in RIPA buffer (150 mM NaCl, 50 mM Tris-HCl (pH 8), 1% NP-40, 0.1% SDS, 0.5% deoxycholate acid containing protease inhibitors (mini Complete, Roche)). Samples were divided into two tubes; one was rotated for 4 h at 4° C. with GST-ARTS fusion protein coupled with the glutathione beads, or GST-XIAP fusion protein. The second tube was incubated with glutathione beads alone (negative control). Samples were centrifuged at 4000-RPM at 4° C. for 4 minute and washed five times in lysis buffer, then boiled in sample buffer for 5 minutes to elute bound proteins Proteins were separated on 12.5% SDS-PAGE gel followed by Western blot analysis using monoclonal anti-ARTS antibody (Sigma), monoclonal anti-XIAP antibody (BD Transduction Laboratories) or monoclonal anti-myc antibody (Clontech).

In Vitro Binding Assay

Recombinant ARTS protein was generated with the TNT-Quick Coupled Transcription/Translation System™ (Promega) and incubated overnight at 4° C. with either recombinant GST-XIAP bound to glutathione beads, or with GST alone bound to glutathione beads, followed by Western blot with anti-ARTS or anti-GST antibody (Gibco).

### Results

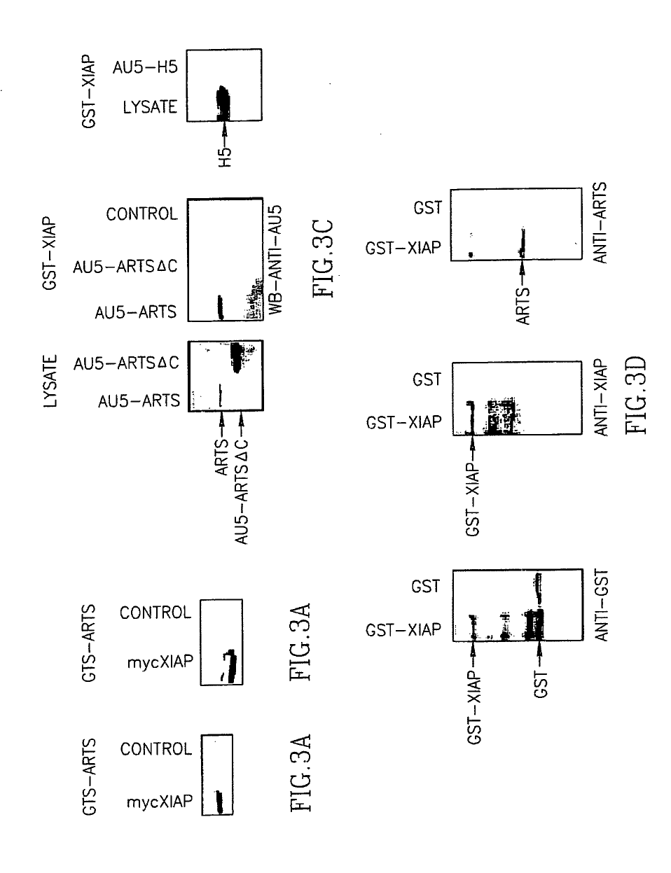

One possible target for the pro-apoptotic action of Peanut/ARTS are the Inhibitor of Apoptosis Proteins (IAPs), which act immediately downstream of Reaper, Hid and Grim to inhibit caspases. In order to explore this possibility, the ability of ARTS to physically interact with IAPs was tested. For this purpose, GST pull-down assays were performed on lysates from COS-7 cells that were transiently transfected with pcDNA3 mycXIAP, using GST-ARTS. Western blot analysis with anti-XIAP and anti-Myc showed that XIAP bound to ARTS (FIG. 3A-B). To confirm these results, the reverse pull-down experiment was performed, using GST-XIAP to precipitate ARTS from COS-7 cells transfected with AU5-ARTS. The ARTS protein bound to XIAP (FIG. 3C), confirming the previous result.

In order to test whether the ARTS-XIAP interaction was direct, GST pull-down assays were performed with GST-XIAP and purified recombinant ARTS protein, followed by Western blot, using anti-ARTS antibody. GST protein alone served as a negative control. The ARTS-XIAP binding was shown to be direct (FIG. 3D).

The findings of this Example show that ARTS protein and XIAP interact, and that the interaction is direct.

### Example 6

### The Unique 68 Amino Acid C-Terminal Sequence of ARTS is Necessary for Interaction with XIAP

In order to ascertain whether the unique 68 amino acid C-terminal sequence of ARTS was necessary for interaction with XIAP, cells transfected with AU5-ARTSΔC or AU5-H5 were included as additional groups in the GST-XIAP experiment described above. The AU5-ARTSΔC construct lacks the 68 amino acids in the unique C′-terminal sequence of ARTS. The H5 septin protein is derived by differential splicing from the same locus as ARTS, and the two proteins share most exons, but H5 is a considerably larger protein that lacks pro-apoptotic activity (Larisch S et al, Nat Cell Biol 2: 915-921, 2000). Neither ARTSΔC nor H5 were able to bind to XIAP (FIG. 3C), demonstrating that the unique 68 amino acid C-terminal sequence of ARTS is necessary for interaction with XIAP.

### Example 7

### Interaction of ARTS with XIAP Occurs in Un-Transfected Cells, and Correlates with Apoptosis

### Methods

Co-Immunoprecipitation

Protein extracts were prepared with lysis buffer (150 mM NaCl, 50 mM Tris-HCl (pH 8), 1% NP-40, 0.5% deoxycholate acid) with protease inhibitors. Lysates containing equal amounts of total protein were pre-cleared overnight with 1 mg mouse IgG (Sigma) coupled with protein A/G-Sepharose mix (Amersham Biosciences) and the beads centrifuged. Supernatants were immuno-precipitated using 5 μl of anti-ARTS antibody for 4 h, anti-XIAP antibody, rabbit anti-myc antibody, or mouse IgG (negative control). Protein A/G Sepharose beads were added for 1 hour and washed four times with PBS. Antibodies against XIAP (as above), Myc (Santa Cruz), or anti-ARTS (NT) antibodies directed against the common N′ terminus of ARTS and H5 (ProSci Incorporated) were used for Western blot.

Apoptosik Assays

Apoptotic cells were detected using the anti-H2A.X antibody (Upstate; Paull, et al., 2000) or TUNEL. For TUNEL assays, COS-7 cells were transiently transfected with AU5-ARTS, AU5-ARTSΔC, AU5-ARTSmGTP and AU5-H5. Twenty-four hours after transfection, the cells were treated with staurosporine (STS) (1 mM) for 1 or 3 hours, then fixed and permeabilized. Apoptosis levels were determined using the TUNEL. In situ™ cell death detection TMR kit (Roche) according to the manufacturer's protocol. All slides were coded and experiments were performed in a blind manner.

### Results

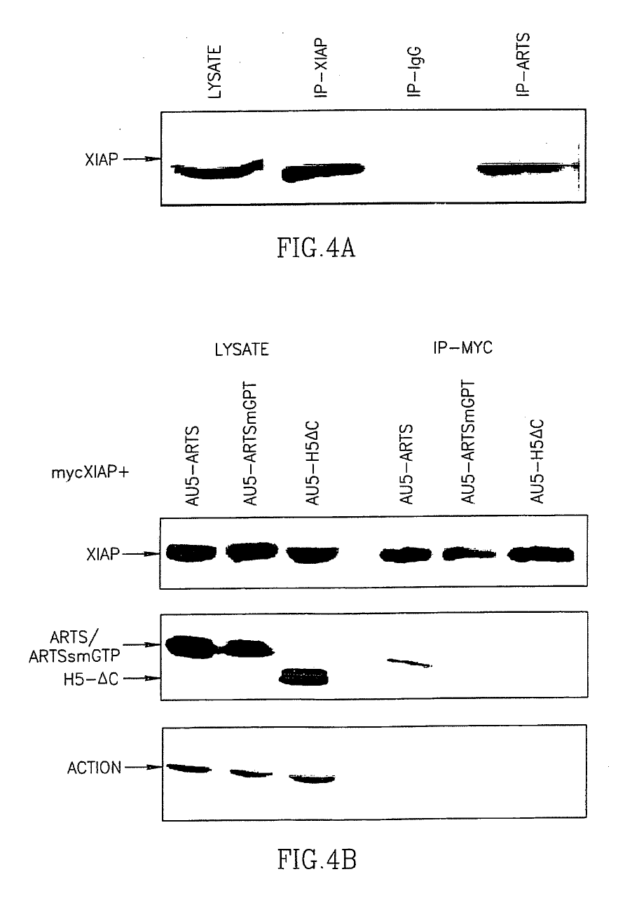

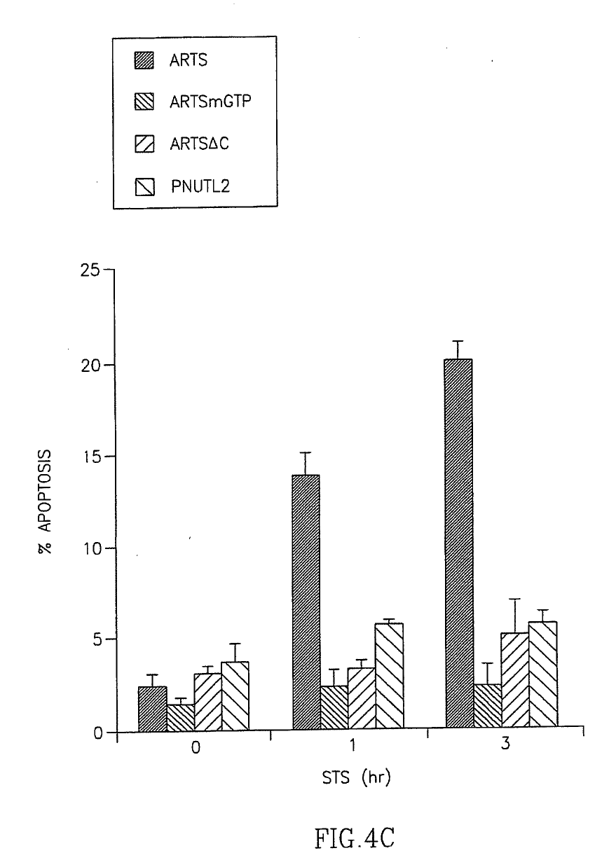

The next experiment tested whether endogenous ARTS and XIAP interact in un-transfected cells. For this purpose, apoptosis was induced in COS-7 cells using staurosporine, and ARTS-protein complexes were immuno-precipitated using an anti-ARTS antibody coupled to protein G/A sepharose beads was. Western blots analysis of these complexes revealed the presence of XIAP (FIG. 4A). These findings demonstrate that in otherwise un-manipulated apoptotic cells, endogenous ARTS and XIAP proteins interact with each other.

H5 and ARTSmGTP (a mutant with an inactivated GRP-binding site) are ARTS mutants that cannot induce TGF-β-mediated apoptosis. Neither of these mutants bound to XIAP in a co-IP (co-immunoprecipitation) assay (FIG. 4B). In addition, ARTS mutants ARTSΔC and PNUTL2, which did not bind XIAP, also did not induce apoptosis (FIG. 4C).

Thus, binding of ARTS to XIAP is specific and highly correlated with its apoptotic function. The above findings show that the ARTS-XIAP complex plays an important role in induction apoptosis.

In apoptotic cells, which do not over-express ARTS, only low levels of diffuse XIAP-staining were seen. In contrast, in cells over-expressing ARTS, XIAP was found at significantly higher levels in the nucleus. Thus, it appears that ARTS may be responsible for XIAP translocation to the nucleus.

### Example 8

### Immunofluorescence Confirms the Existence of ARTS-XIAP Complexes in Apoptotic Cells

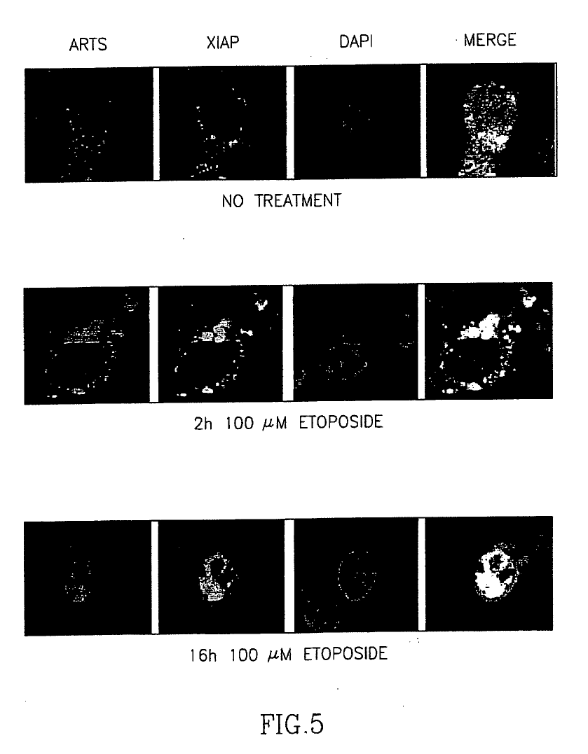

To confirm the finding that ARTS-XIAP complexes exist in apoptotic cells, COS-7 cells transfected with AU5-ARTS and pcDNA3-Myc-XIAP were visualized by confocal microscopy. Apoptosis was induced with etoposide, and cells were stained after 2 and 16 hours. In non-apoptotic cells, ARTS and XIAP had distinct, essentially non-overlapping distributions, with ARTS primarily localized to mitochondria, whereas XIAP was localized to the cytoplasm, with some peri-nuclear concentration (FIG. 5, top panel). In contrast, two hours after induction of apoptosis there was extensive overlap between ARTS and XIAP staining in aggregates that were most prominent in the vicinity of the nucleus (FIG. 5, middle panel). After 16 hours of induction, most of the staining for both proteins was confined to the nucleus.

Thus, a striking level of co-localization of ARTS and XIAP occurs in apoptotic cells. These findings demonstrate that once ARTS is released from mitochondria in response to apoptotic stimuli, it binds rapidly and efficiently to XIAP, and both proteins remain in a complex subsequently,

### Example 9

### Formation of ARTS-XIAP Complexes in Apoptotic Cells Does Not Require Caspase Activation

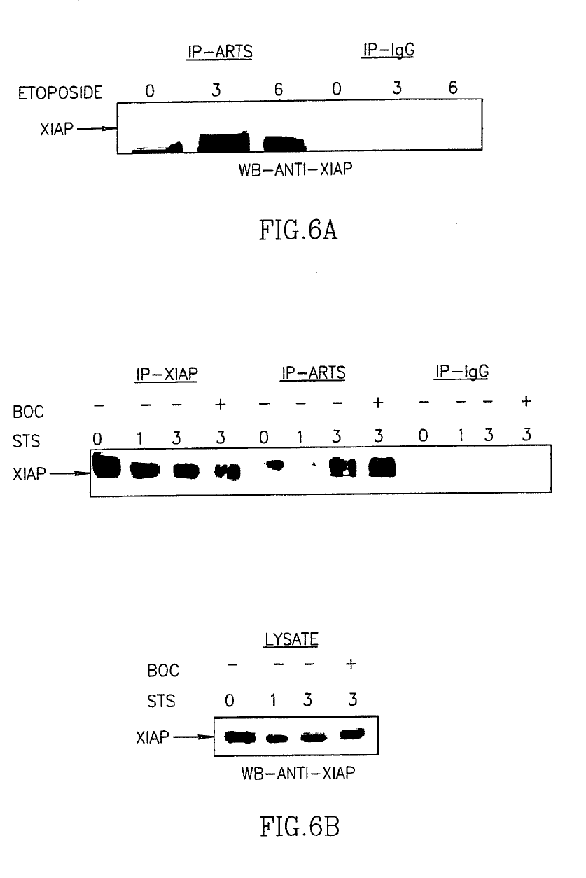

Co-IP was used to further study interactions between endogenous ARTS and XIAP in response to apoptotic stimuli. For initial experiments, NRP154 cells, which express high levels of endogenous ARTS, were used. Apoptosis was induced with etoposide, and ARTS-XIAP binding was assessed after 3 and 6 hours, using the monoclonal antibody described above to detect XIAP (FIG. 6A). Prior to treatment, only a small number of ARTS-XIAP complexes were detected, presumably due to a small amount of cells undergoing apoptosis without treatment. In contrast, after three hours of etoposide treatment, a large number of ARTS-XIAP complexes were detected; this amount increased further after 6 hours of apoptotic induction.

The co-IP experiments were then repeated with COS-7 cells, which express very low levels of endogenous ARTS. COS-7 cells were treated with staurosporine for 1 and 3 hours to induce apoptosis. Thereafter, lysates were prepared, and ARTS-XIAP complexes were detected by co-IP/Western blot analysis (FIG. 6B). As with NRP154 cells, very few ARTS-XIAP complexes were observed without apoptotic induction. After three hours of incubation with staurosporine, there was a significant increase in ARTS-XIAP binding. Thus, apoptosis induces formation of ARTS-XIAP complexes in multiple cell types.

It was next ascertained whether caspase activity and/or execution of apoptosis are required to release ARTS from mitochondria and allow interaction with XIAP, To test this possibility, the co-IP experiments were repeated in the presence of the broad-spectrum caspase inhibitor BOC. Treatment of BOC did not inhibit, and in fact slightly increased, formation of ARTS-XIAP complexes (FIG. 6B). Therefore, ARTS-XIAP interactions do not depend on caspase activity and do not occur as a consequence of apoptosis. Taken together, these results demonstrate that the induction of apoptosis promotes interaction between ARTS and XIAP in a caspase-independent mechanism. The mechanism is likely to depend on release of ARTS protein from mitochondria.

### Example 10

### Induction of Apoptosis Decreases XIAP Protein Levels

### Materials and Experimental Methods

Constructs

Stably transfected COS-7 pE-ARTS cells were generated using the pEF1-IRES vector (Hobbs S et al, Biochem Biophys Res Commun 252: 368-372, 1998). The construct contains an EF-1 promoter followed by a multi cloning site, an EMC (Encephalomyocarditis virus) internal ribosome entry sites (IRES), and a puromycin resistance gene. AU5-tagged ARTS was inserted into the XhoI site downstream of the EF-1 promoter. The construct was stably transfected into COS-7 cells. The cells were maintained in medium containing 4 μg/ml puromycin.

Proteasome Inhibition

Cells were treated for 2 hours with 20 mM of the proteasome inhibitor (MG132, Calbiochem) prior to induction of apoptotis.

### Results

In the above experiment, a decrease in XIAP protein levels was readily detectable after one hour (FIG. 6C). Since this reduction of XIAP protein was not sensitive to the caspase inhibitor BOC, it was not simply the consequence of caspase activation and/or apoptosis.

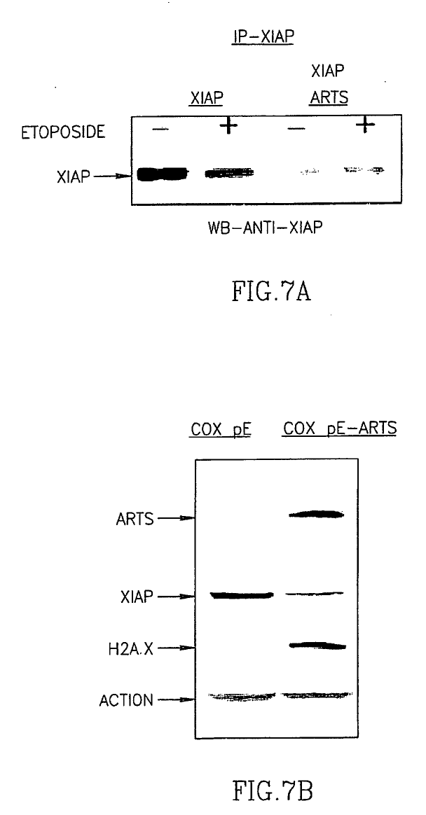

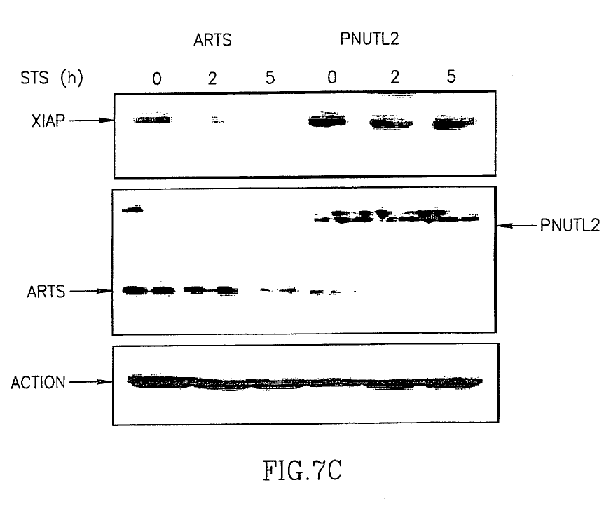

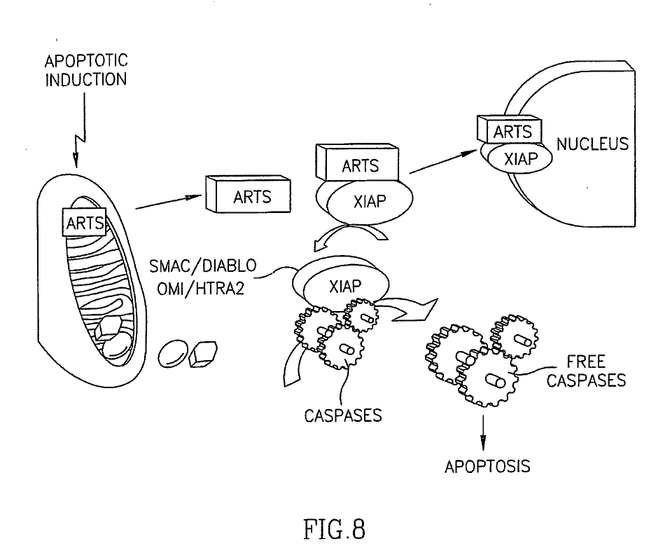

The mechanism of down-regulation of XIAP was further studied in cells in which ARTS protein was over-expressed. COS-7 cells were transiently transfected with pcDNA3-myc-XIAP and AU5-ARTS, and XIAP levels were assessed both with anti-Myc and anti-XIAP antibodies. The amount of XIAP was significantly decreased in cells over-expressing ARTS compared to cells transfected with XIAP alone (FIG. 7A, see also FIG. 6B for endogenous levels of ARTS). Similar results were obtained in COS-7 cells stably transfected with an ARTS construct (FIG. 7B). These cells, which over-express high levels of ARTS, show high levels of the apoptotic marker H2A.X that is detected only in apoptotic cells.

Furthermore, treatment of cells with the proteasome inhibitor MG132 blocked ARTS-induced reduction of XIAP levels, indicating that proteasome-mediated protein degradation is involved in ARTS-mediated down-regulation of XIAP (data not shown). In addition, COS-7 cells transiently transfected with ARTS and treated with staurosporine had reduced XIAP protein levels, while cells transfected with a non-apoptotic related septin (PNUTL2) showed no effect (FIG. 7C)

The findings of this Example show that ARTS reduces XIAP protein levels via proteasome-mediated protein degradation.

In conclusion, the above findings show that ARTS promotes apoptosis through binding to and inhibiting an anti-apoptotic function of XIAP, as depicted in FIG. 8: In living cells, ARTS is localized to mitochondria. Upon receiving an apoptotic stimulus, ARTS is released from mitochondria by a caspase-independent mechanism and binds XIAP, reducing XIAP protein levels. In one embodiment, XIAP protein levels are reduced by proteasome-mediated degradation. In another embodiment, XIAP protein levels are reduced by translocation of ARTS-XIAP complexes to the nucleus. As a result of down-regulation of XIAP levels, caspase activity becomes de-repressed, and apoptosis is facilitated.

### Example 11

### Identification of the Region of ARTS Protein Necessary to Bind and Inactivate XIAP

### Methods

A series of constructs is generated, expressing mutant ARTS proteins with progressive deletions from the N-terminus and C-terminus, using the method of Pues H et al (Nucleic Acids Research, 25 (6): 1303-1304, 1997). A number of constructs are created with the GTP-binding domain mutant described above in combination with deletion mutants. GST binding assays, Co-IP assays, and apoptosis assays are performed as described above.

### Results

In order to define the region of ARTS involved in binding to and inactivating XIAP, constructs expressing a series of N-terminal-deleted ARTS proteins are generated. The ability of the mutant ARTS proteins to bind XIAP is tested by producing the mutant proteins in vitro, and assaying binding to GST-XIAP as described above. Positive results in the GST binding assay are confirmed by expression in COS-7 cells and co-IP with co-expressed myc-tagged XIAP protein, as described above.

Next, a series of C-terminal deleted mutant ARTS proteins containing the largest N-terminal deletion found to have XIAP-binding activity is generated, and their XIAP-binding activity is tested. In addition, the point mutations inactivating the GTP binding motif are introduced into selected N-terminal and C-terminal mutant ARTS proteins. The XIAP-binding activity of these mutant proteins is tested as described above.

Next, the constructs that bind XIAP are tested for their ability to down-regulate XIAP and induce apoptosis. The constructs are expressed in COS-7 cells, and levels of XIAP and induction of apoptosis are measured as described above. In select samples, immuno-fluorescence is used to ascertain whether expression of the mutant ARTS proteins results in transduction of XIAP to the nucleus. Results are confirmed in A549, K562 and HL-60 cells.

At least part of the 68 amino acids unique ARTS C′-terminal sequence is found to be necessary for XIAP binding and inactivation and for induction of apoptosis. In addition, a functional GTP binding motif is found to be required for optimum activity in at least some of the deletion constructs.

### Example 12

### Design of ARTS Protein Non-Peptide Mimetics

### Methods

The biologically active region of ARTS, as delineated in Example 11, is used as the basis for generation of ARTS mimetics with apoptosis-inducing activity, using a method known in the art, for example, one of the methods described in one of the following references: (Song J et al, Biochem Cell Biol 76(2-3): 177-188, 1998; Vogt A et al, J Biol. Chem. 270(2): 660-4, 1995; Alexopoulos K et al, J Med Chem 47(13): 3338-52,2004; Andronati S A et al, Curr Med Chem 11(9): 1183-211, 2004; and Breslin M J et al, Bioorg Med Chem Lett 13(10): 1809-12, 2003).

### Results

Non-peptide ARTS mimetics are tested for ability to bind XIAP in vitro and in vivo, and to cause down-regulation of XIAP and induction of apoptosis in cells, using the methods described above, to identify compounds with therapeutic promise in treating apoptosis-related disorders. Compounds exhibiting these properties and having acceptable toxicity profiles are tested in animal models of apoptosis-related disorders such as cancer.

1. A method of identifying a domain or region of an ARTS (Apoptosis Related Protein in the TGF-β Signaling Pathway) protein that mediates a biological activity, said method comprising the steps of:
generating a subset of test fragments of said ARTS protein that bind to an IAP (Inhibitors of Apoptosis Proteins) protein; and
analyzing said subset for inclusion of a polypeptide common to all of said test fragments in said subset,
whereby said polypeptide defines a domain or region of an ARTS protein that mediates a biological activity.

2. The method of claim 1, wherein said ARTS protein has a sequence as set forth in SEQ ID No: 6.

3. The method of claim 1, wherein said biological activity is an apoptosis, a killing of a cancer cell, or an activity against a psoriasis, a tuberculosis infection, a Bartonella infection, a vitiligo, an atopic dermatitis, a hyper-proliferative or UV-responsive dermatosis, or a lymphohistiocytosis.

4. The method of claim 1, wherein said biological activity is a binding to an IAP protein.

5. The method of claim 4, wherein said IAP protein is an XIAP protein.

6. The method of claim 1, wherein said biological activity is a release of a caspase from an IAP protein.

7. The method of claim 6, wherein said IAP protein is an XIAP protein.

8. The method of claim 1, wherein said IAP protein has a sequence as set forth in SEQ ID No: 7.

9-10. (canceled)

11. The method of claim 1, wherein said LAP protein is an X-link inhibitor of apoptosis protein (XIAP).

12. A method of inducing an apoptosis or a killing of a cancer cell, comprising administering a compound resulting from the method of claim 1.

13-14. (canceled)

15. A method of identifying a compound useful for a chemotherapy of a neoplastic disease or disorder, comprising testing the mimetic compound of claim 9 for an activity against said neoplastic disease or disorder, whereby, if said mimetic compound exhibits an activity against said neoplastic disease or disorder, then said mimetic compound is a compound useful for a chemotherapy of a neoplastic disease or disorder.

16-17. (canceled)

18. The method of claim 15, wherein said neoplastic disease or disorder comprises a decreased level or function of said ARTS protein.

19. The method of claim 15, wherein said neoplastic disease or disorder comprises an increased level or function of the IAP protein of claim 1.

20. A method of identifying a domain or region of an ARTS (Apoptosis Related Protein in the TGF-β Signaling Pathway) protein that mediates a biological activity, said method comprising the steps of:
generating a subset of test fragments of said ARTS protein that reduce a level of an IAP (Inhibitors of Apoptosis Proteins) protein; and
analyzing said subset for inclusion of a polypeptide common to all of said test fragments in said subset,
whereby said polypeptide defines a domain or region of an ARTS protein that mediates a biological activity.

21. The method of claim 20, wherein said ARTS protein has a sequence as set forth in SEQ ID No: 6.

22. The method of claim 20, wherein said biological activity is an apoptosis, a killing of a cancer cell, or an activity against a psoriasis, a tuberculosis infection, a Bartonella infection, a vitiligo, an atopic dermatitis, a hyper-proliferative or UV-responsive dermatosis, or a lymphohistiocytosis.

23. The method of claim 20, wherein said biological activity is a binding to an IAP protein.

24. The method of claim 23, wherein said IAP protein is an XIAP protein.

25. The method of claim 20, wherein said biological activity is a release of a caspase from an LIP protein.

26. The method of claim 25, wherein said LAP protein is an XIAP protein.

27. The method of claim 20, wherein said IAP protein has a sequence as set forth in SEQ ID No: 7.

28-29. (canceled)

30. The method of claim 20, wherein said LAP protein is an X-Link Inhibitor of Apoptosis (XIAP) protein.

31. A method of inducing an apoptosis or a killing of a cancer cell, comprising administering a compound resulting from the method of claim 20.

32-33. (canceled)

34. A method of identifying a compound useful for a chemotherapy of a neoplastic disease or disorder, comprising testing the mimetic compound of claim 28 for an activity against said neoplastic disease or disorder, whereby, if said mimetic compound exhibits an activity against said neoplastic disease or disorder, then said mimetic compound is a compound useful for a chemotherapy of a neoplastic disease or disorder.

35-38. (canceled)

39. A method of identifying a domain or region of an ARTS (Apoptosis Related Protein in the TGF-β Signaling Pathway) protein that mediates a biological activity, said method comprising the steps of:
generating a subset of test fragments of said ARTS protein that induce a translocation of an IAP protein; and
analyzing said subset for inclusion of a polypeptide common to all of said test fragments in said subset,
whereby said polypeptide defines a domain or region of an ARTS protein that mediates a biological activity.

40-46. (canceled)

47. The method of claim 39, wherein said translocation is a nuclear translocation.

48. A mimetic compound of the common region identified by the method of claim 39.

49. The mimetic compound of claim 48, wherein said mimetic compound is a non-peptide compound.

50. (canceled)

51. A method of inducing an apoptosis or a killing of a cancer cell, comprising administering the mimetic compound of claim 48.

52. A method of treating: a psoriasis, a tuberculosis infection, a Bartonella infection, a vitiligo, an atopic dermatitis, a hyper-proliferative or UV-responsive dermatosis, or a lymphohistiocytosis, comprising administering the mimetic compound of claim 48.

53. (canceled)

54. A method of identifying a compound useful for a chemotherapy of a neoplastic disease or disorder, comprising testing the mimetic compound of claim 48 for an activity against said neoplastic disease or disorder, whereby, if said mimetic compound exhibits an activity against said neoplastic disease or disorder, then said mimetic compound is a compound useful for a chemotherapy of a neoplastic disease or disorder.

55-56. (canceled)

57. The method of claim 54, wherein said neoplastic disease or disorder comprises a decreased level or function of said ARTS protein.

58. The method of claim 54, wherein said neoplastic disease or disorder comprises an increased level or function of the IAP protein of claim 39.

59. A method of inducing apoptosis in a cell, comprising the step of inhibiting or reducing a degradation of an ARTS (Apoptosis Related Protein in the TGF-β Signaling Pathway) protein.

60. The method of claim 59, whereby said degradation is mediated by a proteasome.

61. The method of claim 59, wherein said ARTS protein has a sequence as set forth in SEQ ID No: 6.

62. An isolated complex, comprising an ARTS (Apoptosis Related Protein in the TGF-β Signaling Pathway) protein and an IAP (Inhibitors of Apoptosis Proteins) protein.

63. The isolated complex of claim 62, wherein said ARTS protein has a sequence as set forth in SEQ ID No: 6.

64. The isolated complex of claim 62, wherein said IAP protein is an X-Link Inhibitor of Apoptosis (XLAP) protein.

65. The isolated complex of claim 62, wherein said IAP protein has a sequence as set forth in SEQ ID No: 7.

66. A compound that selectively binds to the isolated complex of claim 62.

67. A compound that inhibits a formation of the isolated complex of claim 62.

68. A compound that disrupts the isolated complex of claim 62.

69. A method of testing a compound for an ability to inhibit or reduce an incidence of an apoptosis, comprising determining whether said compound disrupts the isolated complex of claim 62, whereby if said compound disrupts the isolated complex of claim 62, then said compound inhibits or reduces an incidence of an apoptosis.

70-80. (canceled)

81. A method of testing a compound for an ability to inhibit or reduce an incidence of an apoptosis, comprising determining whether said compound prevents a binding of an ARTS (Apoptosis Related Protein in the TGF-β Signaling Pathway) protein to an XIAP (X-link inhibitor of apoptosis) protein, whereby if said compound prevents binding of said ARTS protein to said IAP protein then said compound inhibits or reduces an incidence of an apoptosis.

82. A method of treating or reducing an incidence of a disease or disorder, comprising administering a compound that prevents a binding of an ARTS (Apoptosis Related Protein in the TGF-β Signaling Pathway) protein to an XIAP (X-link inhibitor of apoptosis) protein, thereby treating or reducing an incidence of a neuro-degenerative disease.

83. The method of claim 82, wherein said disease or disorder is a neuro-degenerative disease, an ischemic injury, a myelodysplasia, or an atherosclerosis.

84-86. (canceled)

87. A method of treating or reducing an incidence of an auto-immune disease, comprising administering a compound that prevents a binding of an ARTS (Apoptosis Related Protein in the TGF-β Signaling Pathway) protein to an XIAP (X-link inhibitor of apoptosis) protein, thereby treating or reducing an incidence of an auto-immune disease.

88-92. (canceled)

93. The method of claim 87, wherein said auto-immune disease is a Crohn's disease, an ulcerative colitis, a cytopenia, a pancreatitis, or a periodontitis.
